SEQUENCE LISTING (36 sequences)

SEQ ID NO: 1 - DNA, 19 bp, artificial sequence
  synthetic oligonucleotide
cctcgtcttggccttttgg

SEQ ID NO: 2 - DNA, 20 bp, artificial sequence
  synthetic oligonucleotide
catcttctaccggcatcttc

SEQ ID NO: 3 - DNA, 20 bp, artificial sequence
  synthetic oligonucleotide
aaaggctgagaggaactacc

SEQ ID NO: 4 - DNA, 18 bp, artificial sequence
  synthetic oligonucleotide
accagccttctcctctgc

SEQ ID NO: 5 - DNA, 20 bp, artificial sequence
  synthetic oligonucleotide
gtgttacgtcgcccttgatt

SEQ ID NO: 6 - DNA, 20 bp, artificial sequence
  synthetic oligonucleotide
tgaaagagggctggaagaga

SEQ ID NO: 7 - DNA, 18 bp, artificial sequence
  synthetic oligonucleotide
gtgtcacccacaacgtgc

SEQ ID NO: 8 - DNA, 18 bp, artificial sequence
  synthetic oligonucleotide
agggccacatagcacagc

SEQ ID NO: 9 - DNA, 23 bp, artificial sequence
  synthetic oligonucleotide
gctgaaagcagaaagagattatc

SEQ ID NO: 10 - DNA, 21 bp, artificial sequence
  synthetic oligonucleotide
tggagttcttctcttctggag

SEQ ID NO: 11 - DNA, 19 bp, artificial sequence
  synthetic oligonucleotide
gaagtcctagccagtctcc

SEQ ID NO: 12 - DNA, 21 bp, artificial sequence
  synthetic oligonucleotide
cagcttgagatatgtgtcacc

SEQ ID NO: 13 - DNA, 21 bp, artificial sequence
  synthetic oligonucleotide
gccaagaagacatccctgaag

SEQ ID NO: 14 - DNA, 18 bp, artificial sequence
  synthetic oligonucleotide
gggtccctcgactcctac

SEQ ID NO: 15 - DNA, 20 bp, artificial sequence
  synthetic oligonucleotide
gcacagcagtccaacgtaga

SEQ ID NO: 16 - DNA, 20 bp, artificial sequence
  synthetic oligonucleotide
tctccaaatgggatctctgg

SEQ ID NO: 17 - DNA, 23 bp, artificial sequence
  synthetic oligonucleotide
catgttccagtatgactccactc

SEQ ID NO: 18 - DNA, 20 bp, artificial sequence
  synthetic oligonucleotide
ggcctcaccccatttgatgt

SEQ ID NO: 19 - DNA, 20 bp, artificial sequence
  synthetic oligonucleotide
gagcctcatgaaagcaggac

SEQ ID NO: 20 - DNA, 20 bp, artificial sequence
  synthetic oligonucleotide
gaagttctgaggtggcaagc

SEQ ID NO: 21 - DNA, 20 bp, artificial sequence
  synthetic oligonucleotide
gagttgcaacctctttgtgc

SEQ ID NO: 22 - DNA, 21 bp, artificial sequence
  synthetic oligonucleotide
caggtgtgtaaccaatgatcc

SEQ ID NO: 23 - DNA, 23 bp, artificial sequence
  synthetic oligonucleotide
gactctggtgatttcttgctaac

SEQ ID NO: 24 - DNA, 20 bp, artificial sequence
  synthetic oligonucleotide
caccatggtctcttgagacg

SEQ ID NO: 25 - DNA, 20 bp, artificial sequence
  synthetic oligonucleotide
cgtcgtgatccccacttact

SEQ ID NO: 26 - DNA, 21 bp, artificial sequence
  synthetic oligonucleotide
gaacacacagggtttgccttc

SEQ ID NO: 27 - DNA, 20 bp, artificial sequence
  synthetic oligonucleotide
gacagctttctgcaactcgg

SEQ ID NO: 28 - DNA, 22 bp, artificial sequence
  synthetic oligonucleotide
cttgtggacatatccacagagg

SEQ ID NO: 29 - DNA, 22 bp, artificial sequence
  synthetic oligonucleotide
gcaatgcagacgtagtgatcag

SEQ ID NO: 30 - DNA, 20 bp, artificial sequence
  synthetic oligonucleotide
ccttctttcctccaacgtcc

SEQ ID NO: 31 - DNA, 20 bp, artificial sequence
  synthetic oligonucleotide
cttctgcaactccgacatcg

SEQ ID NO: 32 - DNA, 18 bp, artificial sequence
  synthetic oligonucleotide
cctgtcagcaggtactgg

SEQ ID NO: 33 - DNA, 25 bp, artificial sequence
  synthetic oligonucleotide
caaggatattcagtatgtctacacg

SEQ ID NO: 34 - DNA, 22 bp, artificial sequence
  synthetic oligonucleotide
ctggtggtagtgatgattcagg

SEQ ID NO: 35 - DNA, 20 bp, artificial sequence
  synthetic oligonucleotide
actcctatccccaccttcca

SEQ ID NO: 36 - DNA, 20 bp, artificial sequence
  synthetic oligonucleotide
aagatccgtaggaggccaat

(19) United States
(12) Patent Application Publication
(10) Pub. No.: US 2016/0166651 A1
(43) Pub. Date: Jun. 16, 2016

(54) SYSTEMIC DELIVERY OF VIRUS VECTORS ENCODING UROCORTIN-2 AND RELATED GENES TO TREAT DIABETES-RELATED CARDIAC DYSFUNCTIONS AND CONGESTIVE HEART FAILURE

(71) Applicant: The Regents of the University of California, Oakland, CA (US)

(72) Inventors: H. Kirk HAMMOND, La Jolla, CA (US); Mei Hua GAO, San Diego, CA (US)

(21) Appl. No.: 14/678,796

(22) Filed: Apr. 3, 2015

Related U.S. Application Data
(60) Provisional application No. 61/974,662, filed on Apr. 3, 2014.

(51) Int. Cl. A61K 38/22; C07K 14/575

(52) U.S. Cl. CPC A61K 38/2228; C07K 14/57509; A61K 48/00

(57) ABSTRACT

In alternative embodiments, provided are methods for treating, ameliorating or protecting (preventing) congestive heart failure (CHF) or a diabetes-related cardiac dysfunction, comprising: providing a urocortin 2-encoding and/or a urocortin 3-encoding nucleic acid, transcript or message, or gene, operatively linked to a transcriptional regulatory sequence, optionally contained in an expression vehicle or a vector such as an adeno-associated virus (AAV), e.g., an AAV8 serotype; and administering to an individual or a patient in need thereof, such as a type 2 diabetic (T2DM), e.g., by IV administration, thereby treating, ameliorating or protecting against (preventing) the T2DM and/or the diabetes-related cardiac dysfunction in the individual or patient.

### RELATED APPLICATIONS

This application claims the benefit of priority to U.S. Provisional Patent Application Serial No. (USSN) 61/974,662, filed Apr. 3, 2014. The aforementioned application is expressly incorporated herein by reference in its entirety and for all purposes.

### STATEMENT AS TO RIGHTS TO INVENTIONS MADE UNDER FEDERALLY SPONSORED RESEARCH

This invention was made with government support under grant nos. 306402 (HK066941), P01 HL66941, HL088426, HL081741, and HL107200; and, P01 HL066941-11A1, awarded by the National Institutes of Health (NIH), DHHS; and I01 BX001515 and 1101bBX000783, Veteran's Administration (VA) Merit Grants. The government has certain rights in the invention.

### TECHNICAL FIELD

This invention relates to generally to cellular and molecular biology, gene therapy and medicine and more specifically to compositions and methods for treating, ameliorating or protecting (preventing) an individual or a patient with a type 2 diabetes (T2DM) who also has a diabetes-related cardiac dysfunction.

### BACKGROUND

Despite numerous drugs and other therapies type 2 diabetes (T2DM) affects millions of patients including 35% of those with congestive heart failure (CHF). It is a major risk for the development of coronary and peripheral artery disease and, consequently, with myocardial infarction, CHF and stroke. Sustained hyperglycemia is also independently associated with abnormal cardiac function. Eventually insulin is the central therapy for treatment, but drugs that increase insulin sensitivity and preserve beta cell function play a pivotal role in early management. However, many oral T2DM drugs have adverse effects in subjects with CHF, and are associated with weight gain.

### SUMMARY

In alternative embodiments, provided are methods for treating, ameliorating or protecting (preventing) an individual or a patient with a congestive heart failure (CHF), or an individual with a type 2 diabetes (T2DM) who also has a diabetes-related cardiac dysfunction, comprising: providing a urocortin 2 (UCn-2)-encoding, urocortin 1 (UCn-1)-encoding, and/or a urocortin 3 (UCn-3)-encoding nucleic acid, transcript or message, or gene, operatively linked to a transcriptional regulatory sequence; or an expression vehicle, a vector, a recombinant virus, or equivalent, having contained therein a urocortin 2-encoding and/or a urocortin 3-encoding nucleic acid, transcript or message, or gene, operatively linked to a transcriptional regulatory sequence, and the expression vehicle, vector, recombinant virus, or equivalent can express the urocortin 2-encoding and/or a urocortin 3-encoding nucleic acid, gene, transcript or message in a cell or in vivo; and administering or delivering the urocortin 2-encoding and/or a urocortin 3-encoding nucleic acid, gene, transcript or message operatively linked to a transcriptional regulatory sequence, or the expression vehicle, vector, recombinant virus, or equivalent, to an individual or a patient in need thereof, thereby treating, ameliorating or protecting against (preventing) the type 2 diabetes and diabetes-related cardiac dysfunction in the individual or patient. Provided are compositions and in vitro and ex vivo methods.

In alternative embodiments, provided are methods for treating, ameliorating or protecting (preventing), slowing the progress of, or reversing, an individual or a patient having:

a congestive heart failure (CHF);

a type-2 diabetes mellitus (T2DM) and congestive heart failure (CHF); and/or

an individual or a patient having a Type 2 diabetes mellitus and a diabetes-related cardiac dysfunction.

In alternative embodiments, provided are method for treating, ameliorating or protecting (preventing), slowing the progress of, or reversing: a congestive heart failure (CHF); a type-2 diabetes mellitus (T2DM) and congestive heart failure (CHF); or a Type 2 diabetes mellitus and a diabetes-related cardiac dysfunction; in an individual or a patient comprising:

(a) (i) providing a urocortin 2 and/or a urocortin 3 polypeptide-encoding nucleic acid or gene operatively linked to a transcriptional regulatory sequence; or an expression vehicle, a vector, a recombinant virus, or equivalent, having contained therein a urocortin 2 and/or a urocortin 3-encoding nucleic acid or gene, or a urocortin 2 and/or a urocortin 3 polypeptide-expressing nucleic acid, transcript or message, and the expression vehicle, vector, recombinant virus, or equivalent can express the urocortin 2 and/or a urocortin 3-encoding nucleic acid, gene, transcript or message in a cell or in vivo; and

(ii) administering or delivering the urocortin 2 and/or a urocortin 3 polypeptide-encoding nucleic acid, gene, transcript or message operatively linked to a transcriptional regulatory sequence, or the expression vehicle, vector, recombinant virus, or equivalent, to the cell, or an individual or a patient in need thereof,

thereby treating, ameliorating or protecting (preventing), slowing the progress of, or reversing, the: congestive heart failure (CHF); the type-2 diabetes mellitus (T2DM) and congestive heart failure (CHF); or the Type 2 diabetes mellitus and diabetes-related cardiac dysfunction, in the individual or patient, or thereby treating, ameliorating (including slowing the progress of), reversing or protecting against (preventing) the individual or patient against the Type 2 diabetes and/or related heart disease (diabetes-related cardiac dysfunction);

(b) the method of (a), wherein the expression vehicle, vector, recombinant virus, or equivalent is or comprises:

an adeno-associated virus (AAV), a lentiviral vector or an adenovirus vector,

an AAV serotype AAV5, AAV6, AAV8 or AAV9,

a rhesus-derived AAV, or the rhesus-derived AAV AAVrh.10hCLN2,

an AAV capsid mutant or AAV hybrid serotype,

an organ-tropic AAV mutant, optionally liver-tropic or skeletal muscle-tropic,

wherein optionally the AAV is engineered to increase efficiency in targeting a specific cell type that is non-permissive to a wild type (wt) AAV and/or to improve efficacy in infecting only a cell type of interest,

and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;

(c) the method of (a), wherein the urocortin 2-encoding and/or a urocortin 3-encoding nucleic acid, gene, transcript or message is operatively linked to a regulated or inducible transcriptional regulatory sequence;

(d) the method of (c), wherein the regulated or inducible transcriptional regulatory sequence is a regulated or inducible promoter,

wherein optionally a positive (an activator) and/or a negative (a repressor) modulator of transcription and/or translation is operably linked to the urocortin 2-, urocortin 1-, and/or a urocortin 3 polypeptide-encoding nucleic acid, gene, transcript or message;

(e) the method of any of (a) to (d), wherein administering the urocortin 2-, urocortin 1-, and/or a urocortin 3 polypeptide-encoding nucleic acid, gene, transcript or message operatively linked to a transcriptional regulatory sequence, or the expression vehicle, vector, recombinant virus, or equivalent, to an individual or a patient in need thereof results in a urocortin 2 and/or a urocortin 3 protein being released into the bloodstream or general circulation, or an increased or sustained expression of the urocortin 2 and/or a urocortin 3 protein in the cell,

wherein optionally the release or increased or sustained expression of the urocortin 2 and/or a urocortin 3 protein is dependent on activation of an inducible promoter, or de-repression of a repressor, operably linked to the urocortin 2 and/or a urocortin 3 polypeptide-encoding nucleic acid, gene, transcript or message; or

(f) the method of any of (a) to (e), wherein the Type 3 diabetes and diabetes-related cardiac dysfunction is clinically responsive to the increased urocortin 2 and/or a urocortin 3 polypeptide level in vivo, and optionally a cardiac contractile dysfunction or a congestive heart failure (CHF) is treated, ameliorated, improved or prevented.

In alternative embodiments of exemplary methods of the invention:

(a) the urocortin 2 and/or a urocortin 3 nucleic acid, transcript or gene operatively linked to the transcriptional regulatory sequence; or the expression vehicle, vector, recombinant virus, or equivalent, is administered or delivered to the individual or a patient in need thereof, by oral, intramuscular (IM) injection, by intravenous (IV) injection, by subcutaneous (SC) or intradermal injection, by intrathecal injection, by intra-arterial (IA) injection, by intracoronary injection, by inhalation, or by a biolistic particle delivery system, or by using a “gene gun”, air pistol or a HELIOS™ gene gun (Bio-Rad Laboratories, Hercules, Calif.); or

(b) the urocortin 2 and/or a urocortin 3-encoding nucleic acid, transcript or gene operatively linked to the transcriptional regulatory sequence; or the expression vehicle, vector, recombinant virus, or equivalent, is administered or delivered to the individual or a patient in need thereof, by introduction into any tissue or fluid space within the body that is adjacent to or is drained by the bloodstream, such that the encoded protein may be secreted from cells in the tissue and released into the bloodstream.

In alternative embodiments, the methods further comprise administering, or co-administering, a nucleic acid, transcript or gene encoding: a mammalian cardiotonic peptide, a growth factor, a Serelaxin, a Relaxin-2, a Brain Natriuretic Peptide, a Prostacyclin Synthase, a Growth Hormone, an Insulin-like Growth Factor-1, or any combination thereof; or, a human cardiotonic peptide, a human growth factor, a Serelaxin, a Relaxin-2, a Brain Natriuretic Peptide, a Prostacyclin Synthase, a Growth Hormone, an Insulin-like Growth Factor-11, or any combination thereof

In alternative embodiments of methods of the invention:

(a) the individual, patient or subject is administered a stimulus or signal that induces expression of the urocortin 2 and/or a urocortin 3-expressing nucleic acid, transcript or gene, or induces or activates a promoter (e.g., operably linked to the urocortin 2 and/or a urocortin 3-expressing nucleic acid, transcript or gene) that induces expression of the urocortin 2 and/or a urocortin 3-expressing nucleic acid, transcript or gene;

(b) the individual, patient or subject is administered a stimulus or signal that induces synthesis of an activator of a promoter, optionally a urocortin 2 and/or a urocortin 3-expressing nucleic acid or gene-specific promoter (e.g., operably linked to the urocortin 2 and/or a urocortin 3-expressing nucleic acid or gene);

(c) the individual, patient or subject is administered a stimulus or signal that induces synthesis of a natural or a synthetic activator of the urocortin 2 and/or a urocortin 3-expressing nucleic acid or gene or the urocortin 2 and/or a urocortin 3-expressing nucleic acid or gene-specific promoter,

wherein optionally the natural activator is an endogenous transcription factor;

(d) the method of (c), wherein the synthetic activator is a zinc-finger DNA binding protein designed to specifically and selectively turn on an endogenous or exogenous target urocortin 2 and/or a urocortin 3 gene, wherein optionally the endogenous target is a urocortin 2 and/or a urocortin 3nucleic acid or gene or an activator of a urocortin 2 and/or a urocortin 3 nucleic acid or gene, or an activator of a promoter operatively linked to a urocortin 2 and/or a urocortin 3-expressing nucleic acid or gene;

(e) the method of any of (a) to (c), wherein the stimulus or signal comprises a biologic, a light, a chemical or a pharmaceutical stimulus or signal;

(f) the individual, patient or subject is administered a stimulus or signal that stimulates or induces expression of a post-transcriptional activator of a urocortin 2 and/or a urocortin 3-expressing nucleic acid or gene, or an activator of a promoter operatively linked to a urocortin 2 and/or a urocortin 3-expressing nucleic acid or gene, or

(g) the individual, patient or subject is administered a stimulus or signal that inhibits or induces inhibition of a transcriptional repressor or a post-transcriptional repressor of a urocortin 2 and/or a urocortin 3-expressing nucleic acid or gene.

In alternative embodiments of methods of the invention: the chemical or pharmaceutical that induces expression of the urocortin 2 and/or a urocortin 3-expressing nucleic acid or gene, or induces expression of the regulated or inducible promoter operatively linked to the urocortin 2 and/or a urocortin 3-expressing nucleic acid or gene, is an oral antibiotic, a doxycycline or a rapamycin; or a tet-regulation system using doxycycline is used to induce expression of the urocortin 2-encoding and/or a urocortin 3-expressing nucleic acid or gene, or an equivalent thereof.

In alternative embodiments of methods of the invention: the urocortin 2-encoding and/or a urocortin 3-expressing nucleic acid or gene or the expression vehicle, vector, recombinant virus, or equivalent, is formulated in a liquid, a gel, a hydrogel, a powder or an aqueous formulation.

In alternative embodiments of methods of the invention: the urocortin 2 and/or a urocortin 3-expressing nucleic acid or gene or the expression vehicle, vector, recombinant virus, or equivalent, or the urocortin 2 and/or a urocortin 3 peptide or polypeptide, is formulated in a vesicle, liposome, nanoparticle or nanolipid particle (NLP) or equivalents, or formulated for delivery using a vesicle, liposome, nanoparticle or nanolipid particle (NLP) or equivalents.

In alternative embodiments of methods of the invention: the urocortin 2 and/or a urocortin 3-expressing nucleic acid or gene or the expression vehicle, vector, recombinant virus, or equivalent, is formulated in, or inserted or transfected into, an isolated or cultured cell, and optionally the cell is a mammalian cell, a cardiac cell, or a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

In alternative embodiments of methods of the invention: the urocortin 2 and/or a urocortin 3-expressing nucleic acid, transcript or gene or the expression vehicle, vector, recombinant virus, or equivalent, or the urocortin 2 and/or a urocortin 3 peptide or polypeptide, is formulated as a pharmaceutical or a sterile formulation.

In alternative embodiments of methods of the invention: the urocortin 2 and/or a urocortin 3-expressing nucleic acid or gene or the expression vehicle, vector, recombinant virus, or equivalent, or the urocortin 2 and/or a urocortin 3 peptide or polypeptide, is formulated or delivered with, on, or in conjunction with a product of manufacture, an artificial organ or an implant.

In alternative embodiments of methods of the invention: the urocortin 2 and/or a urocortin 3-expressing nucleic acid or gene or the expression vehicle, vector, recombinant virus, or equivalent expresses a urocortin 2 and/or a urocortin 3 polypeptide in vitro or ex vivo.

In alternative embodiments provided are methods for treating, ameliorating or protecting (preventing) a Type 2 diabetes related: cardiac contractile dysfunction; congestive heart failure (CHF); cardiac fibrosis; cardiac myocyte disease; dysfunction or apoptosis; and/or, pulmonary hypertension, comprising practicing a method of the invention.

In alternative embodiments, provided are methods of treating, ameliorating or protecting (preventing) a Type 2 diabetes or a pre-diabetes in a patient or an individual comprising:

(a) practicing a method of the invention; and

(b) administering a urocortin-2 (UCn-2) and/or urocortin-3 (UCn-3) peptide or polypeptide, or a nucleic acid, gene, message or transcript encoding a urocortin-2 (UCn-2) and/or urocortin-3 (UCn-3) to an individual or patient in need thereof,

wherein optionally the urocortin-2 (UCn-2) and/or urocortin-3 (UCn-3) peptide or polypeptide is an isolated, a recombinant, a synthetic and/or a peptidomimetic peptide or polypeptide or variant thereof,

thereby treating, ameliorating or protecting (preventing) the diabetes or pre-diabetes in the patient or individual.

In alternative embodiments, provided are uses of:
a urocortin 2 and/or a urocortin 3 polypeptide-encoding nucleic acid or gene operatively linked to a transcriptional regulatory sequence;
an expression vehicle, a vector, a recombinant virus, or equivalent, having contained therein a urocortin 2 and/or a urocortin 3-encoding nucleic acid or gene; or
a urocortin 2 and/or a urocortin 3 polypeptide-expressing nucleic acid, transcript or message, and the expression vehicle, vector, recombinant virus, or equivalent that can express the urocortin 2 and/or a urocortin 3-encoding nucleic acid, gene, transcript or message in a cell or in vivo,

in the manufacture of a medicament, or,

said use being, or comprising:
treating, ameliorating or protecting (preventing), slowing the progress of, or reversing, a type-2 diabetes mellitus (T2DM) and congestive heart failure (CHF) in an individual or a patient,
treating, ameliorating or protecting (preventing), slowing the progress of, or reversing, a cardiac contractile dysfunction; a congestive heart failure (CHF); a cardiac fibrosis; a cardiac myocyte disease, dysfunction or apoptosis; a pulmonary hypertension; a heart, skin, liver, lung, muscle, nerve, brain or kidney disease; or, a hemophilia or a Hemophilia B,
treating, ameliorating or protecting or preventing diabetes or pre-diabetes in a patient or an individual, or
treating, ameliorating or protecting or preventing obesity in a patient or an individual,

wherein optionally the expression vehicle, vector, recombinant virus, or equivalent is or comprises:

an adeno-associated virus (AAV), a lentiviral vector or an adenovirus vector,

an AAV serotype AAV5, AAV6, AAV8 or AAV9,

a rhesus-derived AAV, or the rhesus-derived AAV AAVrh.10hCLN2,

an AAV capsid mutant or AAV hybrid serotype,

an organ-tropic AAV, optionally, liver-tropic or skeletal muscle-tropic,

wherein optionally the AAV is engineered to increase efficiency in targeting a specific cell type that is non-permissive to a wild type (wt) AAV and/or to improve efficacy in infecting only a cell type of interest,

and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;

wherein optionally the urocortin 2 and/or a urocortin 3-encoding nucleic acid, gene, transcript or message is operatively linked to a regulated or inducible transcriptional regulatory sequence;

wherein optionally the regulated or inducible transcriptional regulatory sequence is a regulated or inducible promoter,

wherein optionally a positive (an activator) and/or a negative (a repressor) modulator of transcription and/or translation is operably linked to the urocortin 2 and/or urocortin 3 polypeptide-encoding nucleic acid, gene, transcript or message.

In alternative embodiments, provided are:

urocortin 2 and/or a urocortin 3 polypeptide-encoding nucleic acids or genes operatively linked to a transcriptional regulatory sequence;

expression vehicles, a vector, a recombinant virus, or equivalent, having contained therein a urocortin 2 and/or a urocortin 3-encoding nucleic acid or gene; or

urocortin 2 and/or a urocortin 3 polypeptide-expressing nucleic acids, transcripts or messages,

wherein the expression vehicle, vector, recombinant virus, or equivalent can express the urocortin 2 and/or a urocortin 3-encoding nucleic acid, gene, transcript or message in a cell or in vivo,

for use in the manufacture of a medicament, or,

for use in:
treating, ameliorating or protecting (preventing), slowing the progress of, or reversing, a type-2 diabetes mellitus (T2DM) and congestive heart failure (CHF) in an individual or a patient,
treating, ameliorating or protecting (preventing), slowing the progress of, or reversing, a cardiac contractile dysfunction; a congestive heart failure (CHF); a cardiac fibrosis; a cardiac myocyte disease, dysfunction or apoptosis; a pulmonary hypertension; a heart, skin, liver, lung, muscle, nerve, brain or kidney disease; or, a hemophilia or a Hemophilia B,
treating, ameliorating or protecting or preventing diabetes or pre-diabetes in a patient or an individual, or
treating, ameliorating or protecting or preventing obesity in a patient or an individual,

comprising providing and administering or delivering the:
urocortin 2 and/or a urocortin 3 polypeptide-encoding nucleic acid or gene operatively linked to a transcriptional regulatory sequence;
expression vehicle, a vector, a recombinant virus, or equivalent, having contained therein a urocortin 2 and/or a urocortin 3-encoding nucleic acid or gene; or
urocortin 2 and/or a urocortin 3 polypeptide-expressing nucleic acid, transcript or message, and the expression vehicle, vector, recombinant virus, or equivalent that can express the urocortin 2 and/or a urocortin 3-encoding nucleic acid, gene, transcript or message in a cell or in vivo,

to a cell of the subject, or to a subject in need thereof;

wherein optionally the expression vehicle, vector, recombinant virus, or equivalent is or comprises:

an adeno-associated virus (AAV), a lentiviral vector or an adenovirus vector,

an AAV serotype AAV5, AAV6, AAV8 or AAV9,

a rhesus-derived AAV, or the rhesus-derived AAV AAVrh.10hCLN2,

an AAV capsid mutant or AAV hybrid serotype,

an organ-tropic AAV, optionally, liver-tropic or skeletal muscle-tropic,

wherein optionally the AAV is engineered to increase efficiency in targeting a specific cell type that is non-permissive to a wild type (wt) AAV and/or to improve efficacy in infecting only a cell type of interest,

and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;

wherein optionally the urocortin 2 and/or a urocortin 3-encoding nucleic acid, gene, transcript or message is operatively linked to a regulated or inducible transcriptional regulatory sequence;

wherein optionally the regulated or inducible transcriptional regulatory sequence is a regulated or inducible promoter,

wherein optionally a positive (an activator) and/or a negative (a repressor) modulator of transcription and/or translation is operably linked to the urocortin 2 and/or urocortin 3 polypeptide-encoding nucleic acid, gene, transcript or message.

In alternative embodiments, provided are: methods for treating, ameliorating or protecting (preventing) a congestive heart failure (CHF), or the symptoms of congestive heart failure (CHF), in a subject or individual in need thereof, comprising:

(a) delivering to a subject or individual in need thereof a nucleic acid sequence encoding a urocortin 2 polypeptide,

thereby treating or ameliorating congestive heart failure (CHF) in the subject subject or individual in need thereof;

(b) the method of (a), wherein the nucleic acid sequence is in (e.g., contained within) a vector;

(c) the method of (b), wherein the vector is a viral vector;

(d) the method of (c), wherein the vector is an adeno-associated virus (AAV);

(e) the method of (d), wherein the AAV is a serotype AAV8;

(f) the method of any of (a) to (e), wherein the subject or individual in need thereof has a type 2 diabetes (T2DM);

(g) the method of any of (a) to (f), wherein the nucleic acid sequence is administered by intravenous injection (IV) or intramuscularly.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

FIG. 1A and FIG. 1B illustrate data demonstrating that a single IV injection of AAV8.UCn2 in mice results in a 15-fold increase in plasma UCn2 levels (that persists for at least 7 months¹) and: normalizes glucose utilization via increased insulin sensitivity in two models of type 2 diabetes mice (T2DM) (FIG. 1A) and increases function of the failing heart (FIG. 1B): FIG. 1A graphically illustrates multiple panels of data demonstrating that when normal mice received AAV8.UCn2, IV at a dose of 5×10¹¹ gc, or saline as a negative control, and fed standard chow for 3 weeks (w) and then a high fat diet for 8 w: in the AAV8.UCn2 administered animals improvements were made in glucose levels (“prevention”, “resolution” and “glucose tolerance test”); plasma insulin; and homeostasis model assessment (HOMA-IR), or “insulin resistance”; and, FIG. 1B graphically illustrates data from mice 10 weeks (w) after MI-induced CHF: AAV.UCn2 (5×10¹¹ gc, IV) was delivered (vs saline, the “CHF” column) 5 w after induction of CHF, animal administered the AAV.UCn2 showed improvement in left ventricular (LV) global contractility as measured by Ventricular Contractility Assessment (dP/dt); as discussed in detail in Example 1, below.

FIG. 2 schematically illustrates the protocol for measuring efficacy of AAV8.UCn2-Reg after activation of UCn2 expression in the setting of T2DM and LV dysfunction; as discussed in detail in Example 1, below.

FIG. 3 illustrates a table indicating the beneficial cardiovascular effects of Urocortin-2.

FIG. 4 schematically illustrates how Urocortin-2 (UCn2) interacts with corticotropin releasing factor (CRF) type 2 receptors.

FIG. 5: FIG. 5A Upper Panel schematically illustrates vector map of an exemplary AAV8 vector of the invention, an unregulated expression vector, the chicken beta actin (CBA) promoter circumvents methylation in liver; FIG. 5A Lower Panel graphically illustrates multiple panels of data showing that plasma UCn2 was increased greater than 15-fold 6 weeks (w) after a single IV injection of AAV8.CBA.UCn2, and that liver and LV expression were increased; and, FIG. 5B illustrates schematically illustrates exemplary AAV8 regulated Expression Vectors of the invention for optimized regulated expression systems, these exemplary AAV8 vectors encode regulated expression of mouse UCn2, under tetracycline regulation (Map A) or rapamycin regulation (Map B).

FIG. 6 graphically illustrates multiple panels of data showing LV function in normal mice after IV UCn2 gene transfer; increased systolic and diastolic function in isolated hearts demonstrated an autocrine UCn2 effect after the gene transfer.

FIG. 7 graphically illustrates data of LV calcium (Ca⁺²) handling in normal mice after IV UCn2 gene transfer: FIG. 7A graphically illustrates SERC2a levels after IV UCn2 gene transfer as compared to negative control; FIG. 7B schematically illustrates immunoblotting data showing an increase in P16 phospholamban (PLB) levels after IV UCn2 gene transfer as compared to negative control; FIG. 7C graphically illustrates data showing indo-1 ratio (indo-1 fluorescence ratio) over time in seconds (indo-1 is a fluorescent Ca++ indicator for accurate measurement of intracellular calcium concentrations) after IV UCn2 gene transfer as compared to negative control; FIG. 7D graphically illustrates data showing time to Ca²⁺ decline (t_{1/2}, Tau) after IV UCn2 gene transfer as compared to negative control.

FIG. 8 illustrates two panels of data showing increased function in a failing heart after IV UCn2 gene transfer; including a left schematic illustrating the study protocol; and right graphics graphically illustrate increased LV function after IV UCn2 gene transfer as compared to negative control, measuring LV dP/dt.

FIG. 9 illustrates two panels of data showing effects on blood glucose after IV UCn2 gene transfer; including an upper schematic of the exemplary AAV8 gene transfer vector used, and the lower graphics graphically illustrate fasting glucose and dose-response glucose, where the glucose was assessed 3 to 4 weeks after the gene transfer.

FIG. 10 graphically illustrates multiple panels of data showing the effects of fasting glucose in type 2 diabetes mice (T2DM), showing effects on fasting glucose after IV UCn2 gene transfer in the T2DM mice fed high fat diets (HFD), where normal mice received AAV8.UCn2 vectors (5×10¹¹ gc, IV) or saline as negative control, and standard chow for 3 weeks, then HFD diet for 8 weeks; including glucose levels (“prevention” and “resolution”), glucose tolerance test data, plasma insulin in HFD mice, and pre- and post-administration mice, and homeostasis model assessment (HOMA-IR).

FIG. 11 graphically illustrates the effects of glucose utilization in type 2 diabetes mice (T2DM) after IV UCn2 gene transfer, where db/db mice received AAV8.UCn2 vectors (5×10¹¹ gc, IV) or saline as negative control, and the studies conducted 6 weeks after gene transfer; with left graphic showing glucose levels and right graphic showing area under the curve (AUC); and provides an image of a mouse.

FIG. 12 graphically illustrates the effects of glucose utilization in cultured skeletal muscle cells after IV UCn2 gene transfer, where 200 nM insulin, UCn2 peptide, or both (I+U) are added; cells incubated 60 minutes, and glucose uptake measured.

FIG. 13 graphically illustrates multiple panels of data demonstrating glucose utilization in mice before and (4 to 8 weeks) after receiving AAV8.UCn2, IV at a dose of 5×10¹¹ gc, or saline as a negative control, the graphics showing glucose levels (“prevention”, “resolution” and “glucose tolerance test”); plasma insulin; and homeostasis model assessment (HOMA-IR), or “insulin resistance”.

FIG. 14A schematically illustrates an exemplary AAV8.CBA.UCn2 vector Map and FIG. 14B schematically illustrates the experimental protocol for intravenous administration of the vector; as described in detail in Example 2, below.

FIG. 15A, FIG. 15B, FIG. 15C and FIG. 15D graphically illustrate data demonstrating LV Function in vivo: FIG. 15A and FIG. 15B graphically illustrate data from in vivo studies performed to measure the rate of LV pressure development (LV +dP/dt; A) and decay (LV −dP/dt; B). AAV8.UCn2 increased LV +dP/dt and LV −dP/dt 5 weeks after gene transfer; FIG. 15C and FIG. 15D graphically illustrate data showing that heart rate tended to be higher (FIG. 15D), and LV developed pressure was increased by UCn2 gene transfer (FIG. 15C); as described in detail in Example 2, below.

FIG. 16A, FIG. 16B, FIG. 16C, FIG. 16D and FIG. 16E illustrate cytosolic Ca²⁺ transients in cardiac myocytes from mice with heart failure (HF) after IV AAV8.UCn2 (HF+UCn2) or IV saline: FIG. 16A and FIG. 16B graphically illustrate that basal Ca²⁺ released (systolic-diastolic Ca²⁺) was increased in cardiac myocytes from HF+UCn2 mice (p=0.0001), where FIG. 16A is a representative Indo-1 Ca²⁺ transient recordings from one heart in each group showed increased peak Ca²⁺ in cardiac myocytes isolated from mice with heart failure 5 weeks after UCn2 gene transfer; and, FIG. 16B graphically summarizes data from 3 mice per group are shown; in FIG. 16C and FIG. 16D FIG. 16D, graphically illustrated is time to Ca²⁺ decline (t Tau) was shortened in cardiac myocytes from mice with heart failure 5 weeks after UCn2 gene transfer, and FIG. 16C is a representative normalized Ca²⁺ transients from cardiac myocytes from one heart in each group, and FIG. 16D graphically illustrates summary data from 3 mice per group are shown; and for FIG. 16E graphically illustrates immunoblotting data (top panel) and includes an image of the immunoblot (bottom panel) indicating that UCn2 gene transfer increased SERCA2a protein in LV from normal mice and from mice with heart failure; as described in detail in Example 2, below.

FIG. 17A, FIG. 17B, FIG. 17C and FIG. 17D illustrate Cardiac Myocyte cAMP-PKA Signaling: LV samples (FIG. 17A, FIG. 17) or cardiac myocytes (FIG. 17B) were obtained from mice with heart failure (HF) and from mice with HF that had received AAV8.UCn2 (UCn2); FIG. 17A graphically illustrates cAMP Production; FIG. 17B illustrates an immunoblot showing PKA Activity; FIG. 17C graphically illustrates CamK II Expression and Phosphorylation, where UCn2 gene transfer was associated with reduced Thr286 phosphorylation of CamK II (Left panel, normalized to GAPDH); FIG. 17D graphically illustrates Cardiac Myosin Light Chain Kinase, where UCn2 gene transfer was associated with increased cardiac myosin light chain kinase (cMLCK) protein (Left panel, normalized to GAPDH); as described in detail in Example 2, below.

Like reference symbols in the various drawings indicate like elements.

### DETAILED DESCRIPTION

In alternative embodiments provided are compositions and methods to improve glucose utilization and heart function in subjects with Type 2 diabetes mellitus, or to prevent the onset or occurrence of dysfunctional glucose utilization and heart function in subjects with Type 2 diabetes mellitus. In alternative embodiments provided are compositions, including urocortin-2 (UCn-2) and/or urocortin-3 (UCn-3) expressing nucleic acids, such as vectors, that enables delivery and controlled expression of urocortin-2 (UCn-2) and/or urocortin-3 (UCn-3), resulting in the peptide being released into the bloodstream where it can have beneficial effects on glucose utilization and heart function in subjects with Type 2 diabetes mellitus. In alternative embodiments provided are compositions and methods targeted to a subset of patients with diabetes who have diabetes-related cardiac dysfunction. In alternative embodiments provided are compositions and methods for the treatment of patients with type-2 diabetes and associated cardiac dysfunction to restore euglycemia and improve cardiac function in such patients. In alternative embodiments provided are compositions and methods to treat, ameliorate, reverse, or to prevent the onset or occurrence of, a type-2 diabetes mellitus (T2DM) and a congestive heart failure (CHF) using, e.g., a one-time intravenous (IV) injection of a gene therapy vector, e.g., an adeno-associated virus vector type 8 (AAV8), comprising a nucleic acid encoding a urocortin-2 (UCn-2) and/or a urocortin-3 (UCn-3).

In alternative embodiments, provided are methods practiced on T2DM patients, including the 35% of those T2DM patients with congestive heart failure (CHF). In alternative embodiments, provided are methods practiced to decrease the risk of T2DM patients to develop coronary and peripheral artery disease, myocardial infarction, CHF and/or stroke. In alternative embodiments, provided are methods practiced to treat and/or ameliorate sustained hyperglycemia, which is also independently associated with abnormal cardiac function. In alternative embodiments, provided are methods practiced to increase insulin sensitivity and preserve beta cell function, thus, in alternative embodiments the invention plays a pivotal role in early management of T2DM.

In alternative embodiments, the expression vehicle, e.g., a vector, expressing the gene can be delivered either by intramuscular injection (like a “shot”) or by intravenous injection during an office visit, thereby circumventing the problems encountered when gene expression in the heart itself is required. Sustained secretion of the desired protein in the bloodstream circumvents the difficulties and expense of administering proteins by infusion—which can be particularly problematic for many proteins, which exhibit very short half-lives in the body. In alternative embodiments, provided are for controlled expression of the urocortin-2 (UCn-2) and/or urocortin-3 (UCn-3) expressing nucleic acids, and being able to turn on and turn off gene expression easily and efficiently provides tailored treatment and insures optimal safety.

In alternative embodiments provided are gene transfer compositions and methods to treat, slow the progress of, ameliorate and/or prevent diabetes-related cardiac dysfunction. In alternative embodiments, provided are compositions and methods that can be used with or in place of standard medical therapy for diabetes (usually 3 or more drugs including oral hypoglycemic agents and insulin) and/or standard therapy for heart failure (usually 4 or more drugs). In alternative embodiments, provided are compositions and methods that can be used with or in place of oral hypoglycemic agents, which can have adverse effects in diabetic subjects with cardiac dysfunction. In alternative embodiments, practicing this invention reduces the numbers of medications required by patients, and thereby reduce costs and side effects. In alternative embodiments, practicing this invention can preserve pancreatic beta cell function in diabetes, thereby forestalling the need for insulin.

In exemplary applications, the invention employs a regulated expression system providing for controlled expression of urocortin-2 (UCn-2) and/or urocortin-3 (UCn-3) peptide. For example, the long-term virus expression vector can be injected in a systemic vein (or by intramuscular injection) in a physician's office. Four weeks later, the subject swallows an oral antibiotic (doxycycline or rapamycin), once daily (or less often), which will activate the expression of the gene. The gene is synthesized and released to the subject's blood, and subsequently has favorable physiological effects that benefit glucose utilization and cardiac function in the patient with diabetes-related cardiac dysfunction. When the physician or subject desires discontinuation of the treatment, the subject simply stops taking the activating antibiotic.

To demonstrate the efficacy of an embodiment of the invention, we have used an AAV vector encoding urocortin-2 and administered the vector to mice with CHF using intravenous delivery. The results showed: 1) increased serum levels of the transgene 4-6 weeks after intravenous delivery of the vector; 2) pronounced favorable effects on cardiac contractile function (systolic function); and 3) pronounced favorable effects on cardiac relaxation (diastolic function). In additional studies, to demonstrate the efficacy of an embodiment of the invention, we have shown the usefulness of IV delivery of UCn2 in rodent models of Type 2 diabetes.

In alternative embodiments, provided are expression vehicles, vectors, recombinant viruses and the like for in vivo expression of a urocortin 2-encoding and/or a urocortin 3-encoding nucleic acid or gene to practice the methods of this invention. In alternative embodiments, the expression vehicles, vectors, recombinant viruses and the like expressing the urocortin 2-encoding and/or a urocortin 3-encoding nucleic acid or gene can be delivered by intramuscular (IM) injection, by intravenous (IV) injection, by subcutaneous injection, by inhalation, by a biolistic particle delivery system (e.g., a so-called “gene gun”), and the like, e.g., as an outpatient, e.g., during an office visit.

In alternative embodiments, this “peripheral” mode of delivery, e.g., expression vehicles, vectors, recombinant viruses and the like injected IM or IV, can circumvent problems encountered when genes or nucleic acids are expressed directly in an organ, for example, in liver, skeletal muscle, lung or kidney cells or tissue. Sustained secretion of a desired urocortin 2 and/or a urocortin 3 protein(s) in the bloodstream or general circulation also circumvents the difficulties and expense of administering proteins by infusion.

In alternative embodiments, provided are methods for being able to turn on and turn off urocortin 2-encoding and/or a urocortin 3-expressing nucleic acid or gene expression easily and efficiently for tailored treatments and insurance of optimal safety.

In alternative embodiments, the urocortin 2 and/or a urocortin 3 protein or proteins expressed by the urocortin 2-encoding and/or a urocortin 3-expressing nucleic acid(s) or gene(s) have a beneficial or favorable effects (e.g., therapeutic or prophylactic) on a tissue or an organ, e.g., the heart, blood vessels, lungs, kidneys, or other targets, even though secreted into the blood or general circulation at a distance (e.g., anatomically remote) from their site or sites of action, for example, in alternative embodiments, the urocortin 2 and/or a urocortin 3 protein are expressed in lung, kidney, liver or skeletal muscle tissue, and have a beneficial effect on a remote tissue, e.g., a heart or blood vessel.

In an exemplary embodiment, a urocortin 2-encoding and/or a urocortin 3-expressing nucleic acid or gene encoding Urocortin-2 is used, but other urocortin 2-encoding and/or a urocortin 3-expressing nucleic acids or genes can be used to practice methods of this invention, including but not limited to, e.g., for treating congestive heart failure (CHF) or pulmonary hypertension: Urocortin-3, Brain Natriuretic Peptide (for CHF), Prostacyclin Synthase (for pulmonary hypertension), Growth Hormone, and/or Insulin-like Growth Factor-1, or any combination thereof.

In alternative embodiments provided are applications, and compositions and methods, for a regulated expression system providing for controlled expression of a urocortin 2-encoding and/or a urocortin 3-type gene to treat a heart or lung disease, e.g., congestive heart failure (CHF) or pulmonary hypertension.

For example, in alternative embodiments a recombinant virus (e.g., a long-term virus or viral vector), or a vector, or an expression vector, and the like, can be injected, e.g., in a systemic vein (e.g., IV), or by intramuscular (IM) injection, by inhalation, or by a biolistic particle delivery system (e.g., a so-called “gene gun”), e.g., as an outpatient, e.g., in a physician's office. In alternative embodiments, days or weeks later (e.g., four weeks later), the individual, patient or subject is administered (e.g., inhales, is injected or swallows), a chemical or pharmaceutical that induces expression of the urocortin 2-encoding and/or a urocortin 3-expressing nucleic acids or genes; for example, an oral antibiotic (e.g., doxycycline or rapamycin) is administered once daily (or more or less often), which will activate the expression of the gene. In alternative embodiments, after the “activation”, or inducement of expression (e.g., by an inducible promoter) of the nucleic acid or gene, a urocortin 2 and/or a urocortin 3 protein is synthesized and released into the subject's circulation (e.g., into the blood), and subsequently has favorable physiological effects, e.g., therapeutic or prophylactic, that benefit the individual or patient (e.g., benefit heart, kidney or lung function), depending on the urocortin 2 and/or a urocortin 3 protein or proteins expressed. When the physician or subject desires discontinuation of the treatment, the subject simply stops taking the activating chemical or pharmaceutical, e.g., antibiotic.

The inventors have used an AAV vector encoding Urocortin-2 and administered the vector to mice using intravenous delivery. The results showed: 1) a 17-fold increase in serum levels of the transgene 4-6 weeks after intravenous delivery of the vector; 2) pronounced favorable effects on cardiac contractile function (systolic function); and 3) pronounced favorable effects on cardiac relaxation (diastolic function).

In alternative embodiments, provided are applications comprising: the treatment and improvement of heart function in subjects with Type 2 diabetes mellitus, including treatment of severe, low ejection fraction heart failure; the treatment of pulmonary hypertension; the treatment of heart failure with preserved ejection fraction; replacement of current therapies that require hospitalization and sustained intravenous infusions of vasoactive peptides for the treatment of diabetes-related pulmonary hypertension and heart failure; and, the treatment of other conditions in which controlled expression of a urocortin 2-encoding and/or a urocortin 3-type gene can be used to promote favorable effects at a distance in the body.

### Generating and Manipulating Nucleic Acids

In alternative embodiments, to practice exemplary methods of the invention, provided are isolated, synthetic and/or recombinant nucleic acids or genes encoding urocortin 2-encoding and/or a urocortin 3 polypeptides. In alternative embodiments, to practice the methods of the invention, provided are urocortin 2-encoding and/or a urocortin 3-expressing nucleic acids or genes in recombinant form in an (e.g., spliced into) an expression vehicle for in vivo expression, e.g., in a vector or a recombinant virus. In other alternative embodiments, provided are, e.g., isolated, synthetic and/or recombinant nucleic acids encoding inhibitory nucleic acids (e.g., siRNA, microRNA, antisense, ribozyme) that can inhibit the expression of genes or messages (mRNAs) that inhibit the expression of the desired urocortin 2-encoding and/or a urocortin 3 gene.

In alternative embodiments, nucleic acids of the invention are made, isolated and/or manipulated by, e.g., cloning and expression of cDNA libraries, amplification of message or genomic DNA by PCR, and the like. The nucleic acids and genes used to practice this invention, including DNA, RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides (e.g., urocortin 2 and/or a urocortin 3 chimeric proteins used to practice this invention) generated from these nucleic acids can be individually isolated or cloned and tested for a desired activity. Any recombinant expression system or gene therapy delivery vehicle can be used, including e.g., viral (e.g., AAV constructs or hybrids) bacterial, fungal, mammalian, yeast, insect or plant cell expression systems or expression vehicles.

Alternatively, nucleic acids used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook, ed.,

MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In alternative embodiments, to practice the methods of the invention, urocortin 2-encoding and/or a urocortin 3 fusion proteins and nucleic acids encoding them are used.

In alternative embodiments, a heterologous peptide or polypeptide joined or fused to a protein used to practice this invention can be an N-terminal identification peptide which imparts a desired characteristic, such as fluorescent detection, increased stability and/or simplified purification. Peptides and polypeptides used to practice this invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Nucleic acids or nucleic acid sequences used to practice this invention can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Compounds use to practice this invention include “nucleic acids” or “nucleic acid sequences” including oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Compounds use to practice this invention include nucleic acids, i.e., oligonucleotides, containing known analogues of natural nucleotides. Compounds use to practice this invention include nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Compounds use to practice this invention include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In alternative aspects, compounds used to practice this invention include genes or any segment of DNA involved in producing a urocortin 2-encoding and/or a urocortin 3; it can include regions preceding and following the coding region (leader and trailer) as well as, where applicable, intervening sequences (introns) between individual coding segments (exons). “Operably linked” can refer to a functional relationship between two or more nucleic acid (e.g., DNA) segments. In alternative aspects, it can refer to the functional relationship of transcriptional regulatory sequence to a transcribed sequence. For example, a promoter can be operably linked to a coding sequence, such as a nucleic acid used to practice this invention, if it stimulates or modulates the transcription of the coding sequence in an appropriate host cell or other expression system. In alternative aspects, promoter transcriptional regulatory sequences can be operably linked to a transcribed sequence where they can be physically contiguous to the transcribed sequence, i.e., they can be cis-acting. In alternative aspects, transcriptional regulatory sequences, such as enhancers, need not be physically contiguous or located in close proximity to the coding sequences whose transcription they enhance.

In alternative aspects, the invention comprises use of “expression cassettes” comprising a nucleotide sequences used to practice this invention, which can be capable of affecting expression of the nucleic acid, e.g., a structural gene or a transcript (e.g., encoding a urocortin 2 and/or a urocortin 3 protein) in a host compatible with such sequences. Expression cassettes can include at least a promoter operably linked with the polypeptide coding sequence or inhibitory sequence; and, in one aspect, with other sequences, e.g., transcription termination signals. Additional factors necessary or helpful in effecting expression may also be used, e.g., enhancers.

In alternative aspects, expression cassettes used to practice this invention also include plasmids, expression vectors, recombinant viruses, any form of recombinant “naked DNA” vector, and the like. In alternative aspects, a “vector” used to practice this invention can comprise a nucleic acid that can infect, transfect, transiently or permanently transduce a cell. In alternative aspects, a vector used to practice this invention can be a naked nucleic acid, or a nucleic acid complexed with protein or lipid. In alternative aspects, vectors used to practice this invention can comprise viral or bacterial nucleic acids and/or proteins, and/or membranes (e.g., a cell membrane, a viral lipid envelope, etc.). In alternative aspects, vectors used to practice this invention can include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and can include both the expression and non-expression plasmids. In alternative aspects, the vector used to practice this invention can be stably replicated by the cells during mitosis as an autonomous structure, or can be incorporated within the host's genome.

In alternative aspects, “promoters” used to practice this invention include all sequences capable of driving transcription of a coding sequence in a cell, e.g., a mammalian cell such as a heart, lung, muscle, nerve or brain cell. Thus, promoters used in the constructs of the invention include cis-acting transcriptional control elements and regulatory sequences that are involved in regulating or modulating the timing and/or rate of transcription of a gene. For example, a promoter used to practice this invention can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters used to practice this invention can be those that drive expression continuously under most environmental conditions and states of development or cell differentiation. In alternative embodiments, “Inducible” or “regulatable” promoters used to practice this invention can direct expression of the nucleic acid of the invention under the influence of environmental conditions, administered chemical agents, or developmental conditions.

### Gene Therapy and Gene Delivery Vehicles

In alternative embodiments, methods of the invention comprise use of nucleic acid (e.g., gene or polypeptide encoding nucleic acid) delivery systems to deliver a payload of a urocortin 2-encoding and/or a urocortin 3-encoding nucleic acid or gene, or a urocortin 2-encoding and/or a urocortin 3 polypeptide-expressing nucleic acid, transcript or message, to a cell or cells in vitro, ex vivo, or in vivo, e.g., as gene therapy delivery vehicles.

In alternative embodiments, expression vehicle, vector, recombinant virus, or equivalents used to practice methods of the invention are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. In alternative embodiments, the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see e.g., Wu et al., Mol. Ther. 2006 September; 14(3):316-27. Epub 2006 Jul. 7; Choi, et al., Curr. Gene Ther. 2005 June; 5(3):299-310.

For example, the rhesus-derived AAV AAVrh.10hCLN2 or equivalents thereof can be used, wherein the rhesus-derived AAV may not be inhibited by any pre-existing immunity in a human; see e.g., Sondhi, et al., Hum Gene Ther. Methods. 2012 October; 23(5):324-35, Epub 2012 Nov. 6; Sondhi, et al., Hum Gene Ther. Methods. 2012 Oct. 17; teaching that direct administration of AAVrh.10hCLN2 to the CNS of rats and non-human primates at doses scalable to humans has an acceptable safety profile and mediates significant payload expression in the CNS.

Also, for example, AAV vectors specifically designed for cardiac gene transfer (a cardiotropic AAV) can be used, e.g., the AAVM41 mutant having improved transduction efficiency and specificity in the myocardium, see, e.g., Yang, et al. Virol J. 2013 Feb. 11; 10(1):50.

Because adeno-associated viruses (AAVs) are common infective agents of primates, and as such, healthy primates carry a large pool of AAV-specific neutralizing antibodies (NAbs) which inhibit AAV-mediated gene transfer therapeutic strategies, the methods of the invention comprise screening of patient candidates for AAV-specific NAbs prior to treatment, especially with the frequently used AAV8 capsid component, to facilitate individualized treatment design and enhance therapeutic efficacy; see, e.g., Sun, et al., J. Immunol. Methods. 2013 Jan. 31; 387(1-2):114-20, Epub 2012 Oct. 11.

### Kits and Instructions

Provided are kits comprising compositions and methods of the invention, including instructions for use thereof, including kits comprising cells, expression vehicles (e.g., recombinant viruses, vectors) and the like.

For example, in alternative embodiments, provided are kits comprising compositions used to practice this invention, e.g., comprising a urocortin-2 (UCn-2) peptide or polypeptide; or a urocortin 2-encoding and/or a urocortin 3-encoding nucleic acid, (b) a liquid or aqueous formulation of the invention, or (c) the vesicle, liposome, nanoparticle or nanolipid particle of the invention. In one aspect, the kit further comprising instructions for practicing any methods of the invention, e.g., in vitro or ex vivo methods for increasing a desired urocortin 2-encoding and/or a urocortin 3 level in the bloodstream, or for protecting a cell, e.g., a cardiac or lung cell; or for treating, preventing or ameliorating diabetes or pre-diabetes.

### Formulations

In alternative embodiments, provided are compositions and methods for use in increasing urocortin 2-encoding and/or a urocortin 3 levels in vivo. In alternative embodiments, these compositions comprise urocortin 2-encoding and/or a urocortin 3-encoding nucleic acids formulated for these purposes, e.g., expression vehicles or urocortin 2-encoding and/or a urocortin 3-encoding nucleic acids formulated in a buffer, in a saline solution, in a powder, an emulsion, in a vesicle, in a liposome, in a nanoparticle, in a nanolipoparticle and the like.

In alternative embodiments, provided are methods comprising administration of urocortin 2 and/or a urocortin 3 peptides or polypeptides, or urocortin 2 and/or a urocortin 3-encoding nucleic acids, to treat, ameliorate or prevent a diabetes (including Type 1 and Type 2, or adult onset diabetes) or pre-diabetes, or obesity or excess weight; or to stimulate weight loss, or to act as an appetite suppressant. Accordingly, provided are the appropriate formulations and dosages of urocortin 2 and/or a urocortin 3 peptides or polypeptides, or UCn-2-encoding nucleic acids, for same.

In alternative embodiments, the compositions (including formulations of urocortin 2 and/or a urocortin 3-encoding nucleic acids, can be formulated in any way and can be applied in a variety of concentrations and forms depending on the desired in vitro, in vivo or ex vivo conditions, including a desired in vivo or ex vivo method of administration and the like. Details on techniques for in vitro, in vivo or ex vivo formulations and administrations are well described in the scientific and patent literature.

Formulations and/or carriers of the urocortin 2 and/or a urocortin 3-encoding nucleic acids, or urocortin 2 and/or a urocortin 3 peptides or polypeptides, used to practice this invention are well known in the art. Formulations and/or carriers used to practice this invention can be in forms such as tablets, pills, powders, capsules, liquids, gels, syrups, slurries, suspensions, etc., suitable for in vivo or ex vivo applications.

In alternative embodiments, urocortin 2-encoding and/or a urocortin 3-encoding nucleic acids, or urocortin 2 and/or a urocortin 3 peptides or polypeptides, used to practice this invention can be in admixture with an aqueous and/or buffer solution or as an aqueous and/or buffered suspension, e.g., including a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate. Formulations can be adjusted for osmolarity, e.g., by use of an appropriate buffer.

In practicing this invention, the compounds (e.g., formulations) of the invention can comprise a solution of urocortin 2-encoding, urocortin 1-encoding nucleic acids or genes, or urocortin 2 and/or a urocortin 3 peptides or polypeptides, dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The solutions and formulations used to practice the invention can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent (e.g., urocortin 2-encoding and/or a urocortin 3-encoding nucleic acids or genes) in these formulations can vary widely, and can be selected primarily based on fluid volumes, viscosities and the like, in accordance with the particular mode of in vivo or ex vivo administration selected and the desired results, e.g., increasing in vivo urocortin 2 and/or a urocortin 3 expression.

The solutions and formulations used to practice the invention can be lyophilized; for example, provided are a stable lyophilized formulation comprising urocortin 2-encoding and/or a urocortin 3-encoding nucleic acids or genes, or urocortin 2 and/or a urocortin 3 peptides or polypeptides. In one aspect, this formulation is made by lyophilizing a solution comprising a urocortin 2-encoding, urocortin 1-encoding nucleic acid or gene, or urocortin 2 and/or a urocortin 3 peptides or polypeptides, and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations of the invention can be delivered by the use of liposomes (see also discussion, below). By using liposomes, particularly where the liposome surface carries ligands specific for target cells, or are otherwise preferentially directed to a specific tissue or organ type, one can focus the delivery of the active agent into a target cells in an in vivo or ex vivo application. In alternative embodiments, the target cells are liver, skeletal muscle or liver cells.

### Nanoparticles, Nanolipoparticles and Liposomes

The invention also provides nanoparticles, nanolipoparticles, vesicles and liposomal membranes comprising compounds (e.g., urocortin 2-encoding and/or a urocortin 2-encoding nucleic acids) used to practice the methods of this invention, e.g., to deliver urocortin 2 and/or a urocortin 3 peptides or polypeptides, to an individual, a patient or mammalian cells in vivo or ex vivo. In alternative embodiments, these compositions are designed to target specific molecules, including biologic molecules, such as polypeptides, including cell surface polypeptides, e.g., for targeting a desired cell type, e.g., a mammalian cell such as a skeletal muscle cell or tissue, a liver cell, a kidney cell, a lung cell, a nerve cell and the like.

Provided are multilayered liposomes comprising compounds used to practice this invention, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, e.g., to entrap a urocortin 2-encoding and/or a urocortin 3-encoding nucleic acid or gene.

Liposomes can be made using any method, e.g., as described in Park, et al., U.S. Pat. Pub. No. 20070042031, including method of producing a liposome by encapsulating an active agent (e.g., vectors expressing urocortin 2 and/or a urocortin 3 peptides or polypeptides), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice this invention comprise a substituted ammonium and/or polyanions, e.g., for targeting delivery of a compound (e.g., urocortin 2-encoding and/or a urocortin 3-encoding nucleic acids or genes) used to practice this invention to a desired cell type, as described e.g., in U.S. Pat. Pub. No. 20070110798.

The invention also provides nanoparticles comprising compounds (e.g., urocortin 2-encoding and/or a urocortin 3-encoding nucleic acids or genes, or urocortin 2 and/or a urocortin 3 peptides or polypeptides) used to practice this invention in the form of active agent-containing nanoparticles (e.g., a secondary nanoparticle), as described, e.g., in U.S. Pat. Pub. No. 20070077286. In one embodiment, provided are nanoparticles comprising a fat-soluble active agent of this invention or a fat-solubilized water-soluble active agent to act with a bivalent or trivalent metal salt.

In one embodiment, solid lipid suspensions can be used to formulate and to deliver urocortin 2-encoding and/or a urocortin 3-encoding nucleic acids or genes, or urocortin 2 and/or a urocortin 3 peptides or polypeptides, used to practice the invention to a patient, an individual, or mammalian cell in vivo or ex vivo, as described, e.g., in U.S. Pat. Pub. No. 20050136121.

### Delivery Vehicles

In alternative embodiments, any delivery vehicle can be used to practice the methods or compositions of this invention, e.g., to deliver urocortin 2-encoding and/or a urocortin 3-encoding nucleic acids or genes, or urocortin 2 and/or a urocortin 3 peptides or polypeptides, to practice the methods of the invention in vivo or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate a composition of the invention, wherein a surfactant is associated with a nucleic acid via a non-covalent bond e.g. as described, e.g., in U.S. Pat. Pub. No. 20040151766.

In one embodiment, a nucleic acid or polypeptide used to practice this invention can be applied to cells as polymeric hydrogels or water-soluble copolymers, e.g., as described in U.S. Pat. No. 7,413,739; for example, a nucleic acid or protein can be polymerized through a reaction between a strong nucleophile and a conjugated unsaturated bond or a conjugated unsaturated group, by nucleophilic addition, wherein each precursor component comprises at least two strong nucleophiles or at least two conjugated unsaturated bonds or conjugated unsaturated groups.

In one embodiment, a nucleic acid or protein is applied to cells using vehicles with cell membrane-permeant peptide conjugates, e.g., as described in U.S. Pat. Nos. 7,306,783; 6,589,503. In one aspect, the nucleic acid itself is conjugated to a cell membrane-permeant peptide. In one embodiment, a nucleic acid, protein, and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver a urocortin 2-encoding and/or a urocortin 3-encoding nucleic acids or genes to a cell, e.g., using any electroporation system as described e.g. in U.S. Pat. Nos. 7,109,034; 6,261,815; 5,874,268.

### Products of Manufacture, Implants and Artificial Organs

Provided are products of manufacture comprising cells of the invention (e.g., cells modified to express urocortin 2-encoding and/or a urocortin 3 peptides or polypeptides, to practice the methods of the invention), and use of cells made by methods of this invention, including for example implants and artificial organs, bioreactor systems, cell culture systems, plates, dishes, tubes, bottles and flasks comprising cells modified to express urocortin 2 and/or a urocortin 3 proteins to practice the methods of the invention. Any implant, artificial organ, bioreactor systems, cell culture system, cell culture plate, dish (e.g., petri dish), cell culture tube and/or cell culture flask (e.g., a roller bottle) can be used to practice this invention.

In alternative embodiments provided are a bioreactor, implant, stent, artificial organ or similar device comprising cells modified to express urocortin 2 and/or a urocortin 3 proteins to practice the methods of the invention; for example, including implants as described in U.S. Pat. Nos. 7,388,042; 7,381,418; 7,379,765; 7,361,332; 7,351,423; 6,886,568; 5,270,192; and U.S. Pat. App. Pub. Nos. 20040127987; 20080119909 (describing auricular implants); 20080118549 (describing ocular implants); 20080020015 (describing a bioactive wound dressing); 20070254005 (describing heart valve bio-prostheses, vascular grafts, meniscus implants); 20070059335; 20060128015 (describing liver implants).

### Implanting Cells In Vivo

In alternative embodiments, provided are methods comprising implanting or engrafting cells, e.g., cardiac, lung or kidney cells, comprising or expressing urocortin 2 and/or a urocortin 3-encoding nucleic acids or genes, or urocortin 2 and/or a urocortin 3 peptides or polypeptides, used to practice the invention; and in one aspect, methods of the invention comprise implanting or engrafting the urocortin 2 and/or a urocortin 3-encoding nucleic acids or genes (or cells expressing them), or urocortin-2 (UCn-2) peptides or polypeptides, in a vessel, tissue or organ ex vivo or in vivo, or implanting or engrafting the re-programmed differentiated cell in an individual in need thereof.

Cells can be removed from an individual, treated using the compositions and/or methods of this invention, and reinserted (e.g., injected or engrafted) into a tissue, organ or into the individual, using any known technique or protocol. For example, de-differentiated re-programmed cells, or re-programmed differentiated cells, can be re-implanted (e.g., injected or engrafted) using microspheres e.g., as described in U.S. Pat. No. 7,442,389; e.g., in one aspect, the cell carrier comprises a bulking agent comprising round and smooth polymethylmethacrylate microparticles preloaded within a mixing and delivery system and an autologous carrier comprising these cells. In another embodiment, the cells are readministered to a tissue, an organ and/or an individual in need thereof in a biocompatible crosslinked matrix, as described e.g., in U.S. Pat. App. Pub. No. 20050027070.

In another embodiment, the cells of the invention (e.g., cells made by practicing the methods of this invention) are readministered (e.g., injected or engrafted) to a tissue, an organ and/or an individual in need thereof within, or protected by, a biocompatible, nonimmunogenic coating, e.g., as on the surface of a synthetic implant, e.g., as described in U.S. Pat. No. 6,969,400, describing e.g., a protocol where a cAMP-incompetent AC can be conjugated to a polyethylene glycol that has been modified to contain multiple nucleophilic groups, such as primary amino or thiol group.

In one embodiment, the cells of the invention (e.g., cells made by practicing the methods of this invention) are readministered (e.g., injected or engrafted) to a tissue, an organ and/or an individual in need thereof using grafting methods as described e.g. by U.S. Pat. Nos. 7,442,390; 5,733,542.

Any method for delivering polypeptides, nucleic acids and/or cells to a tissue or organ (e.g., a lung, kidney, liver, skeletal muscle) can be used, and these protocols are well known in the art, e.g., as described in U.S. Pat. No. 7,514,401, describing e.g., using intracoronary (IC), intravenous (IV), and/or local delivery (myocardial injection) of polypeptides, nucleic acids and/or cells to a heart in situ. For example, in alternative embodiments, aerosol drug particles into the lungs and into the bloodstream, gene therapy, continuous infusions, repeated injections and/or sustained release polymers can be used for delivering polypeptides, nucleic acids and/or cells to a tissue or organ (e.g., a lung, kidney, liver, skeletal muscle). In alternative embodiments, nucleic acids and/or cells can be given through a catheter into the coronary arteries or by direct injection into the left atrium or ventricular myocardium via a limited thoracotomy; or delivered into the myocardium via a catheter passed during cardiac catheterization; or delivered into the pericardial space.

In alternative embodiments, nucleic acids or proteins used to practice this invention, or a vector comprising a nucleic acid used to practice the invention (e.g., an AAV, or adenoviral gene therapy vector), or vesicle, liposome, nanoparticle or nanolipid particle (NLP) of the invention, and the like, to a tissue or organ (e.g., a lung, kidney, liver, skeletal muscle); e.g. as described in U.S. Pat. No. 7,501,486.

Compositions used to practice this invention can be used in combination with other therapeutic agents, e.g. angiogenic agents, anti-thrombotic agents, anti-inflammatory agents, immunosuppressive agents, anti-arrhythmic agents, tumor necrosis factor inhibitors, endothelin inhibitors, angiotensin-converting enzyme inhibitors, calcium antagonists, antibiotic agents, antiviral agents and viral vectors.

Compositions used to practice this invention can be used for ameliorating or treating any of a variety of diabetes-related cardiopathies and cardiovascular diseases, e.g., diabetes-related cardiopathies and cardiovascular diseases, e.g., coronary artery disease (CAD); atherosclerosis; thrombosis; restenosis; vasculitis including autoimmune and viral vasculitis such as polyarteritis nodosa, Churg-Strass syndrome, Takayasu's arteritis, Kawasaki Disease and Rickettsial vasculitis; atherosclerotic aneurisms; myocardial hypertrophy; congenital heart diseases (CHD); ischemic heart disease and anginas; acquired valvular/endocardial diseases; primary myocardial diseases including myocarditis; arrhythmias; and transplant rejections; metabolic myocardial diseases and myocardiomyopathies such as congestive, hypertrophic and restrictive cardiomyopathies, and/or heart transplants. In alternative embodiments, compositions used to practice this invention, e.g., urocortin-2 (UCn-2) peptides or polypeptides, are used for treating, ameliorating or protecting (preventing) diabetes or pre-diabetes in a patient or an individual; or suppressing weight gain, or suppressing the appetite, or stimulating or initiating weight loss, in a patient or an individual; or treating, ameliorating or protecting (preventing) diabetes in a patient or an individual.

The invention will be further described with reference to the following examples; however, it is to be understood that the invention is not limited to such examples.

### EXAMPLES

### Example 1

### Intravenous Delivery of AAV8 Encoding Urocortin-2 Increases Cardiac Function in Normal Mice

This example demonstrates the effectiveness of an exemplary embodiment of the invention. In alternative embodiments, provided are compositions and methods for treating and ameliorating type-2 diabetes mellitus (T2DM) and diabetic heart disease using a one-time intravenous (IV) injection of an adeno-associated virus vector serotype-8 (AAV8) encoding urocortin-2 (UCn2), a peptide of the corticotropin releasing factor (CRF) family. In alternative embodiments, the vector (AAV8.UCn2) comprises a regulated expression cassette to enable controlled expression. In alternative embodiments, exemplary vectors are delivered by IV injection, e.g., into a brachial vein during an outpatient visit.

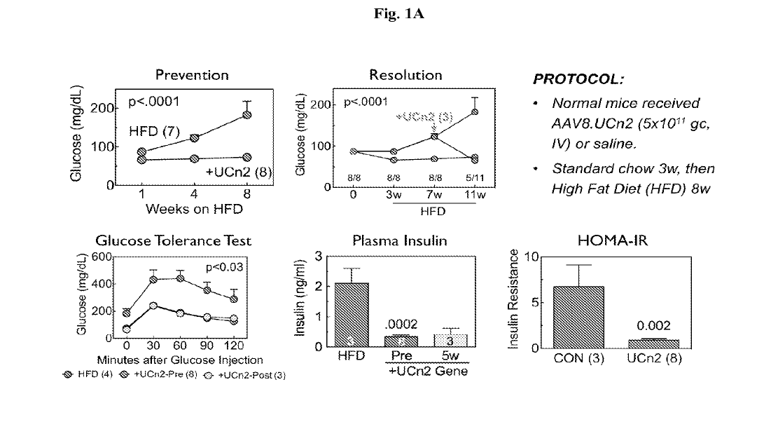

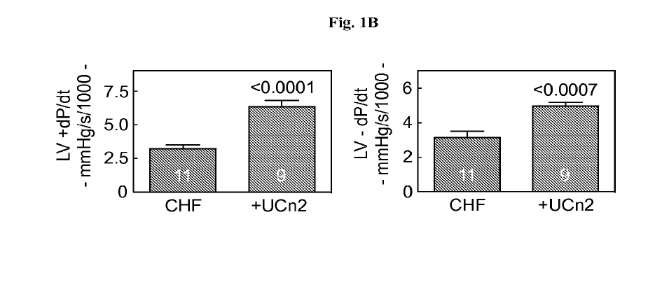

We have demonstrated that a single IV injection of AAV8.UCn2 in mice results in a 15-fold increase in plasma UCn2 levels that persists for at least 7 months¹ and: a) normalizes glucose utilization via increased insulin sensitivity in two models of T2DM (FIG. 1A) and b) increases function of the failing heart (FIG. 1B). In alternative embodiments, methods of the invention comprise IV injection of a vector encoding a peptide with beneficial paracrine effects on insulin sensitivity and cardiac function.

Our data in rodent T2DM indicate that UCn2 gene transfer methods of the invention can: forestall the need for insulin; be well tolerated and beneficial in patients with CHF; not require repeated injections; and, be associated with weight loss.

Methods of this invention, which can have beneficial cardiac effects,¹ can safely be used in subjects with CHF, and will fill an unmet medical need: a novel treatment of T2DM patients with CHF with features not shared by current drugs.

In alternative embodiments, practicing the methods of the invention can forestall the need for insulin, and thus practicing the methods of the invention is beta cell preserving and beneficial to patients with T2DM.

In alternative embodiments practicing the methods of the invention, e.g., using AAV8.UCn2, will reduce rather than increase weight, a problem with current T2DM agents. Because of UCn2's beneficial effects on cardiac function,¹ it can be used safely to treat T2DM patients with CHF, unlike thiazolidinediones.

In alternative embodiments therapies of this invention will be indicated for T2DM subjects with and without CHF and used in place of (or in addition to) oral agents; and can for some patients delay the need for insulin.

In alternative embodiments therapies of this invention focus on early stage T2DM using a transgene that increases insulin sensitivity.

In alternative embodiments therapies of this invention are practiced on subjects with T2DM by administration of vectors, e.g., AAV8.UCn2, IV; where individuals who have failed diet and exercise intervention, and are not yet insulin-dependent may be the ideal candidates. In addition, therapies of this invention can increase function of the normal¹ and failing heart (FIG. 1B), and can in some patients improve function of the failing heart in subjects with T2DM.

In alternative embodiments, IV (intravenous injection) of an AAV8 vector with regulated expression of urocortin-2 will increase glucose utilization and insulin sensitivity, and improve cardiac function in T2DM. As graphically illustrated in FIG. 1A, when normal mice received AAV8.UCn2, IV at a dose of 5×10¹¹ gc, or saline as a negative control, and fed standard chow for 3 weeks (w) and then a high fat diet for 8 w: in the AAV8.UCn2 administered animals improvements were made in glucose levels (“prevention”, “resolution” and “glucose tolerance test”); plasma insulin; and homeostasis model assessment (HOMA-IR), or “insulin resistance”.

In alternative embodiments, therapies of this invention comprise gene transfer, e.g., UCn2, UCn1 and/or UCn3 gene transfer e.g., by intravenous (IV) delivery of a vector, e.g., an AAV vector, encoding a UCn2, UCn1 and/or UCn3 expressing nucleic acid, e.g., a UCn2, UCn1 and/or UCn3 gene or cDNA. In alternative embodiments, systemic vector delivery has an advantage in gene transfer of peptides with paracrine activity as it provides the highest plasma level of transgene for any given AAV dose.

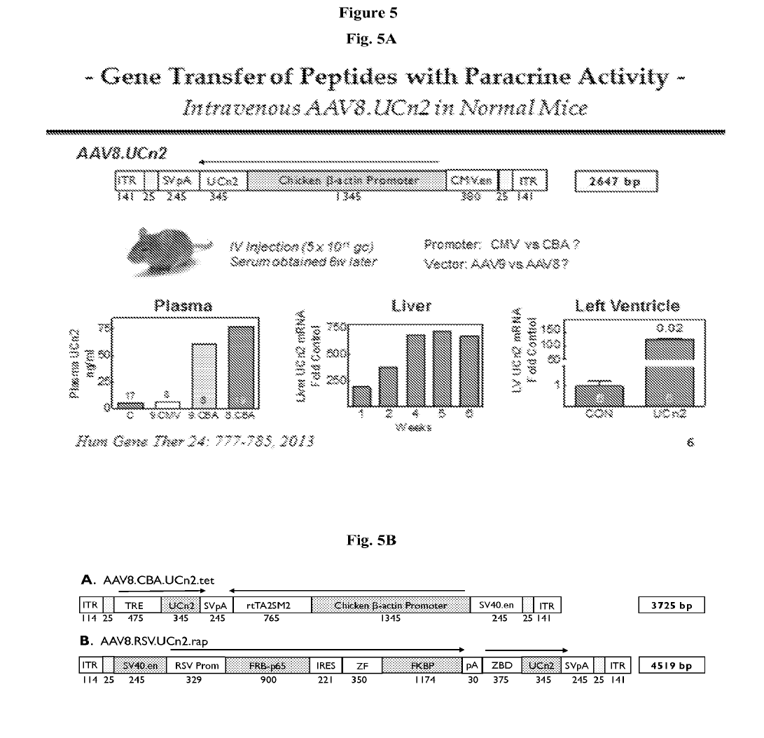

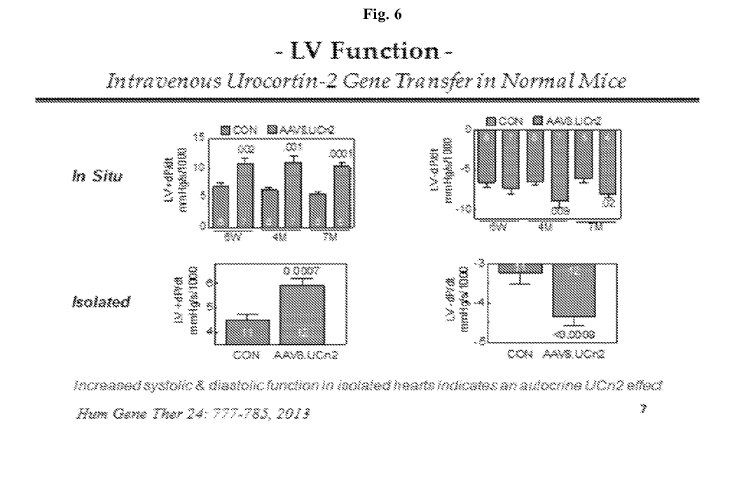

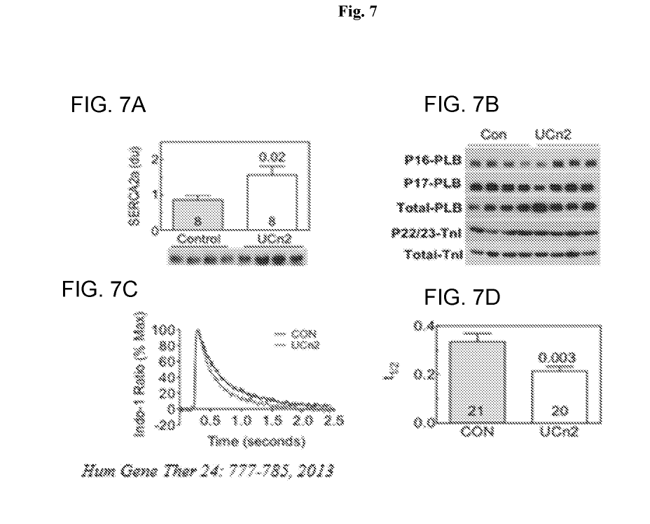

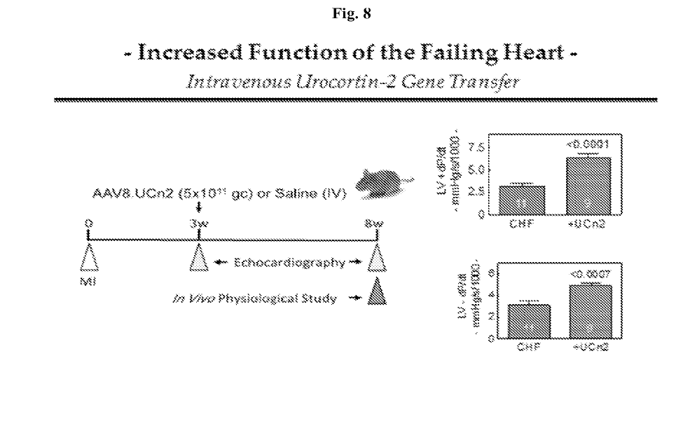

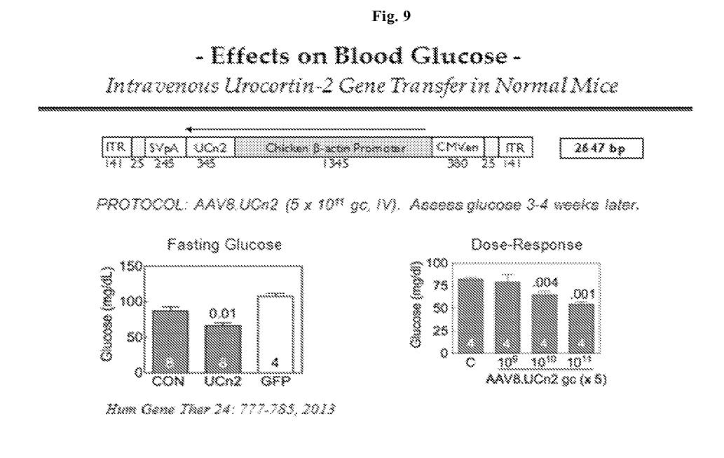

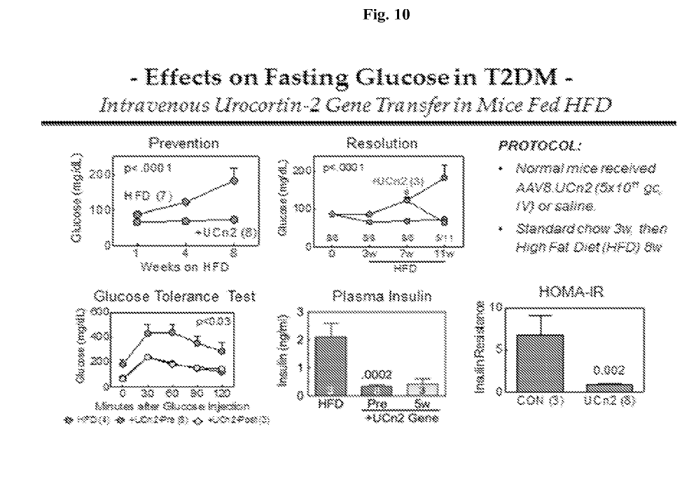

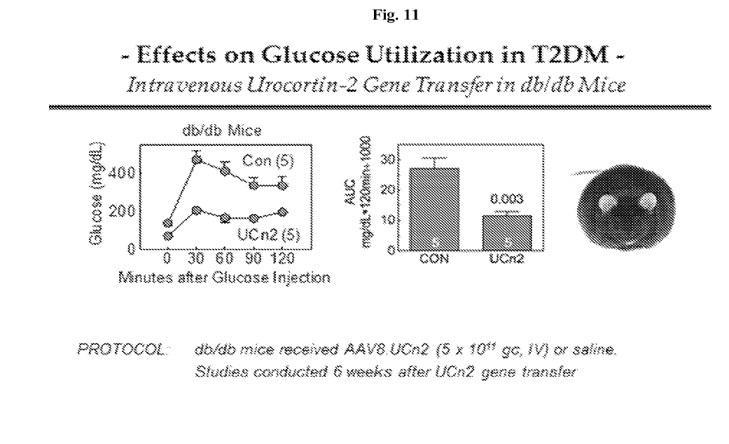

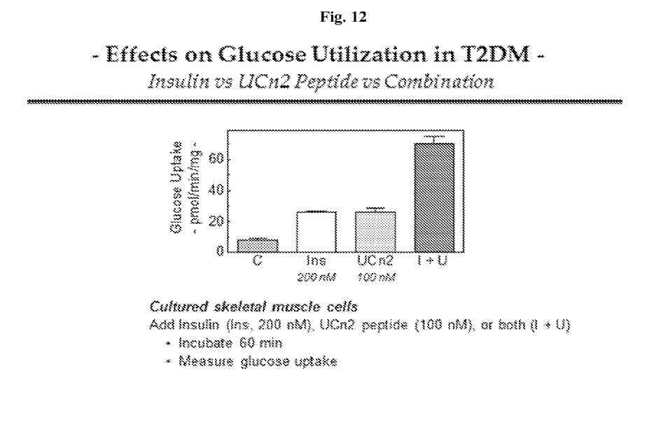

In alternative embodiments, AAV are used, as they can enable longer transgene expression than adenovirus, and avoids insertional mutagenesis associated with retrovirus. Persistent transgene expression has been shown in large animals years after a single injection of AAV vectors. We have confirmed this in mice (see, e.g., FIG. 5) and rats.¹¹ Although recent clinical trials have found that some AAV serotypes incite immune responses after IM injection,¹² newer generation AAV vectors (AAV5, 6, 8 and 9) do not have similar problems in primates.¹³ IV AAV delivery is superior to IM vis-à-vis plasma transgene levels, and AAV5 is superior to AAV5 and AAV6.¹⁴ Pre-existing anti-AAV8 antibodies are not as prevalent in humans (19%) as are other AAV serotypes including AAV1, AAV2 and AAV6 (50-59%).¹⁵ Our data indicate that IV AAV8 is the optimal vector and delivery route to attain sustained increased levels of plasma UCn2 for the proposed studies (FIG. 1).¹

Although robust in striated muscle, the cytomegalovirus (CMV) promoter is susceptible to methylation and inactivation in liver,¹⁶ and our data indicate that promoters less susceptible to methylation are superior. Indeed, although CMV provided a sustained 2.3-fold increase in UCn2 after IV vector delivery, use of the chicken β-actin (CBA) promoter resulted in >15-fold increase in plasma UCn2 (FIG. 1). In alternative embodiments, UCn2, UCn1 and/or UCn3 expressing nucleic acids, e.g., a UCn2, UCn1 and/or UCn3 gene or cDNA, are operatively linked to chicken β-actin (CBA) promoters.

In alternative embodiments, UCn2, UCn1 and/or UCn3 expressing nucleic acids are under “regulated expression”. In alternative embodiments, because of the potential for long-term expression conferred by AAV gene transfer, the ability to turn off expression is desirable in the event that untoward effects develop. In alternative embodiments, regulated expression is used, it can enable the flexibility of intermittent rather than constant transgene delivery. In alternative embodiments, tetracycline and rapamycin regulation systems are used; they have been tested in large animal models.

Data from High Fat Diet (HFD) Model of T2DM.

UCn2 gene transfer both prevented T2DM and treated it once present. Both fasting blood glucose & glucose tolerance tests were normalized. A measure of insulin resistance (HOMA-IR) was reduced. FIG. 1B: Data from mice 10 weeks (w) after MI-induced CHF: AAV.UCn2 (5×10¹¹ gc, IV) was delivered (vs saline) 5 w after induction of CHF. UCn2 gene transfer increased systolic & diastolic LV function (blinded studies).

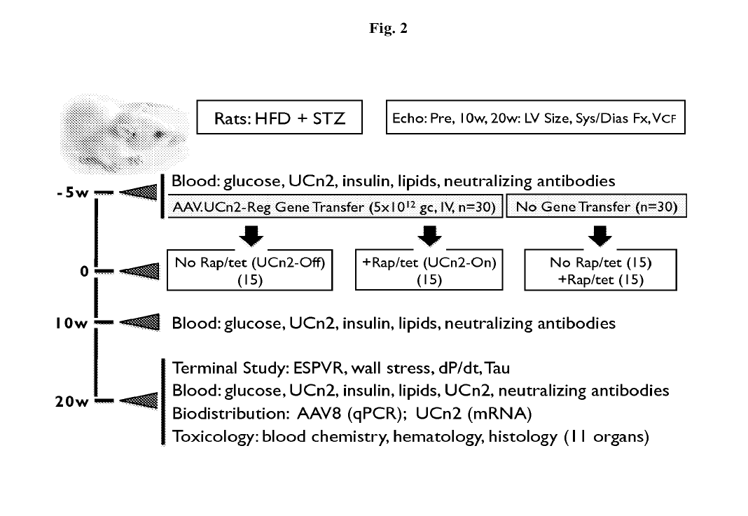

FIG. 2. Test efficacy of AAV8.UCn2-Reg (5×10¹¹ gc, IV) 20 weeks after activation of UCn2 expression in the setting of T2DM & LV dysfunction. We use a model of T2DM associated with abnormal LV systolic & diastolic function that uses high fat diet (HFD) plus streptozotocin (STZ; 35 mg/kg IP×2) in Sprague-Dawley rats.⁷ Serial echocardiography will assess LV size & systolic & diastolic function, including velocity of circumferential fiber shortening (VCF) function. Terminal studies in 15 rats/group are performed using pressure-volume catheters to assess the end-systolic pressure volume relationship (ESPVR), wall stress, rate of LV pressure rise and decay, and Tau. Finally, samples from 11 tissues from each animal undergo biodistribution and toxicology studies; AAV requirements: 1.5×10¹⁴ gc.

Intravenous administration of Urocortin-2 in HFD mice (mice fed high fat diets for ten weeks, then AAV8.UCn2-Reg (5×10¹¹ gc, IV) or IV saline (negative control) at week five, resulted in a 73% reduction in fatty infiltration of the liver, as confirmed by histology analysis.

FIG. 5A. Upper Panel: vector map of unregulated expression vector. CBA promoter circumvents methylation in liver, a problem with CMV. Lower Panel. Plasma UCn2 was increased >15-fold 6 w after a single IV injection of AAV8.CBA.UCn2. Liver and LV expression were increased. Cardiac expression may be important for autocrine effects, which may augment the paracrine effects. Additional data (not shown) document persistent and stable effects on plasma UCn2 and cardiac function 7 months after gene transfer.

FIG. 5B illustrates exemplary regulated Expression Vectors of the invention: for optimal regulated expression systems. These exemplary AAV8 vectors encode regulated expression of mouse UCn2, under Tetracycline regulation (Map A) or Rapamycin regulation (Map B). RSV is used in vector Map B because CBA will not fit with Rap. These two regulated expression vectors will be tested (Aim 1) and the better one selected for Aim 2 & Aim 3 studies. Abbreviations: ITR, inverted terminal repeat; SVpA, polyA from SV40 viral genome (bidirectional); UCn2, urocortin-2; TRE, tetracycline response element; rtTA2SM2, reverse tetracycline controlled transactivator; SV40.en, simian virus 40 enhancer; RSV Prom, Rous sarcoma virus promoter; FRB-p6, part of FRAP, a rapamycin interacting protein, combined with a subunit of transcription factor NF-κB (p65); IRES, internal transcription reentry site; ZF, zinc finger HD1 DNA binding domain; FKBP, FK506 binding protein; pA, minimal polyadenylation segment; ZBD, zinc finger HD DNA binding domain (8 copies)

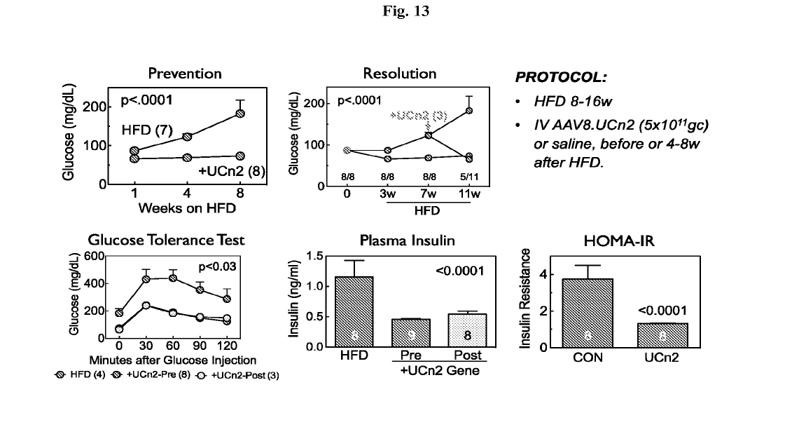

FIG. 13. Left: Data from HFD model of T2DM. UCn2 gene transfer both prevented T2DM (Pre) & treated it once present (Post: gene transfer 4-8 wk after Hyperglycemia present). Both fasting blood glucose & glucose tolerance tests were normalized. A measure of insulin resistance (HOMA-IR) was reduced. Effects confirmed in db/db mice. Above: Data from mice 10 w after MI-induced CHF. AAV.UCn2 (5×10¹¹ gc, IV) was delivered (vs saline) 5 w after CHF, which increased systolic & diastolic LV function (blinded studies).

### Example 2

### Intravenous Delivery of AAV8 Encoding Urocortin-2 Increases Function of the Failing Heart in Mice

This example demonstrates the effectiveness of an exemplary embodiment of the invention, that intravenous delivery of AAV8.UCn2 increases function of the failing heart. In summary, myocardial infarction (MI, by coronary ligation) was used to induce heart failure, which was assessed by echocardiography 3 weeks after MI. Mice with LV ejection fraction (EF)<25% received intravenous delivery of AAV8.UCn2 (5×10¹¹ gc) or saline, and 5 weeks later echocardiography showed increased LV EF in mice that received UCn2 gene transfer (p=0.01). In vivo physiological studies showed a 2-fold increase in peak rate of LV pressure development (LV +dP/dt; p<0.0001) and a 1.6-fold increase in peak rate of LV pressure decay (LV +dP/dt; p=0.0007) indicating increased LV systolic and diastolic function in treated mice. UCn2 gene transfer was associated with increased peak systolic Ca²⁺ transient amplitude and rate of Ca²⁺ decline and increased SERCA2a expression. In addition, UCn2 gene transfer reduced Thr286 phosphorylation of Cam kinase II, and increased expression of cardiac myosin light chain kinase, findings that would be anticipated to increase function of the failing heart. These results demonstrate that a single intravenous injection of AAV8.UCn2 increases function of the failing heart. The simplicity of intravenous injection of a vector encoding a gene with beneficial paracrine effects to increase cardiac function is an attractive clinical strategy.

### Methods

AAV8.UCn2 Vector Production

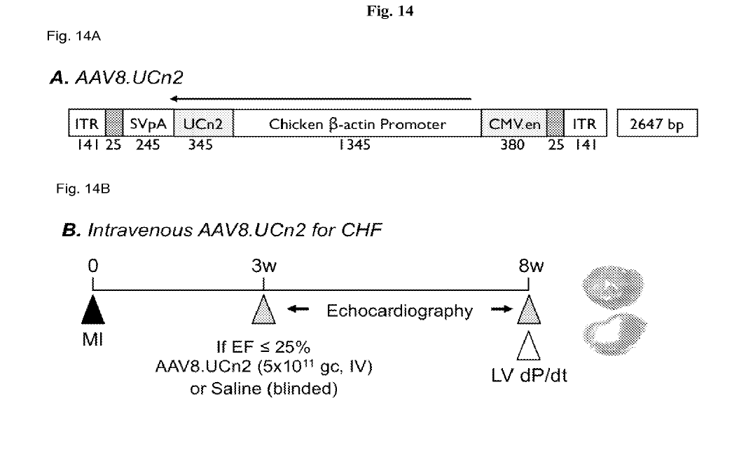

(FIG. 14). A helper virus free AAV8 vector encoding murine urocortin-2 (UCn2) driven by a chicken β-actin (CBA) promoter (AAV8.CBA.UCn2; FIG. 14) was produced by transient transfection of HEK293T cells with the vector plasmid pRep2/Cap8 and pAd-Helper plasmid.²⁸ Plasmid pRep2/Cap8 was obtained from the University of Pennsylvania Vector Core. Cell lysates prepared after 72 hrs of transfection were treated with benzonase and viruses were consolidated through 25% sucrose-cushion ultracentrifugation. The pellets were resuspended for further purification of the virus through anion-exchange column chromatography (Q-Sepharose, GE Health Science) and concentrated by 25% sucrose-cushion ultracentrifugation.^{29,30} Subsequently the pellets were resuspended in 10 mM Tris-HCl (pH 7.9, 1 mM MgCl2, 3% sucrose). Virus titers were determined by real-time qPCR with virus genome DNA prepared from purified virus.

Heart Failure Model

The Animal Use and Care Committee of the VA San Diego Healthcare System approved the studies. Two hundred thirty one male C57BL/6J mice (Jackson Laboratories, Bar Harbor, Me., USA) aged 10-12 weeks, weighing 26.1±0.2 grams were used. We used coronary occlusion to induce large anterior wall MI and CHF as described in detail previously.^{31,32} MI size deliberately was large, approximately 50% of LV, comprising most of the LV free wall (FIG. 14). Consequently, this model is associated with a high initial mortality. Of 231 mice that underwent coronary occlusion, 125 (54%) died before randomization (AAV8.UCn2 or saline) primarily in the first few days after MI. An additional 45 mice (19%) did not show sufficient LV dysfunction 3 weeks after MI to be randomized. Sixty-one mice (26%) had sufficiently low LV ejection fractions (EF<25%) and were randomized, and eleven of these mice died before the final study 5 weeks after randomization: 4 UCn2 (mortality 13%); 7 saline (mortality 23%). The primary end point of was LV function 5 weeks after intravenous delivery of AAV8.UCn2 vs saline in mice with severe heart failure (FIG. 14). Data were acquired and analyzed without knowledge of group identity.

AAV8.UCn2 Delivery

Under anesthesia (1.5% isoflurane via nose cone), a small incision was made to expose the jugular vein for intravenous delivery of AAV8.UCn2 (5×10¹¹ gc in 50 μl) or a similar volume of saline (control).

Effects of UCn2 Gene Transfer on Heart Rate and Blood Pressure

These studies were conducted to assess the effects of UCn2 gene transfer on heart rate and blood in unsedated mice with heart failure. Impaired LV ejection fraction was confirmed 3 weeks after MI, and mice received intravenous AAV8.UCn2 (5×10¹¹ genome copies, gc) or saline. Systolic and diastolic blood pressure and heart rate was measured by tail cuff (Visitech Systems, Apex, N.C.) in unsedated mice.

### Echocardiography.

Echocardiography was performed as previously described.³³ Echocardiography was performed 3 weeks after myocardial infarction to document reduced LV function (EF<25%) and to record LV chamber dimensions. Echocardiographic assessment was then repeated 5 weeks after randomization of mice to receive intravenous delivery of AAV8.UCn2 or saline.

### LV Systolic and Diastolic Function.

Mice were anesthetized with sodium pentobarbital (80 mg/kg, ip) and a 1.4 F conductance-micromanometer catheter (SPR 839, Millar Instruments, Houston, Tex.) was advanced via the right carotid artery across the aortic valve and into the LV cavity. Left ventricular pressure was recorded and stored digitally for processing (IOX1.8 Emka Technologies, Christchurch, Va.) as previously reported.⁶ Subsequently, blood and tissue samples were obtained. After acquisition, the first derivative of LV pressure development (LV +dP/dt) and decline (LV −dP/dt) were used to assess LV systolic and diastolic function. Data were acquired and analyzed without knowledge of group identity.

### Cardiac Myocyte Isolation.

Cardiac myocytes were isolated as previously described.³³

Ca²⁺ Transients.

Cytosolic Ca²⁺ transients were measured using Indo-1 as described previously^{27,34} with modifications. Cardiac myocytes were plated onto laminin-coated glass cover slips and loaded with indo-1/AM (3 μM, Calbiochem, La Jolla Calif.) and dispersing agent, pluronic F-127 (0.02 mg/ml, Calbiochem, La Jolla, Calif.) for 30 min. Following dye loading, cover slips were mounted in a superfusion chamber, rinsed to remove excess indo-1-AM, and mounted on a Nikon Diaphot epifluorescence microscope equipped with a 40× objective interfaced to a Photon Technologies photometry system (Birmingham, N.J.) with the excitation wavelength set to 365 nm via a monochromator. Fluorescence emission was split and directed to two photomultiplier tubes through 20-nm band-pass filters centered at 405 and 485 nm, respectively. The ratio F405/F485 represents a measure for [Ca²⁺]i. During these measurements, cardiac myocytes were superfused with 25 mM HEPES (pH 7.3) containing 2 mM CaCl2. Myocytes were field-stimulated at 0.3 Hz. Ca²⁺ transients were recorded from 144 cardiac myocytes obtained from 6 hearts (3 per group). Diastolic and systolic intracellular Ca²⁺ levels were inferred from the basal and maximal indo-1 ratio per cycle, respectively. Diastolic decay time (tau) was calculated from the normalized Ca²⁺ transient.

Quantitative RT-PCR (qRT-PCR) and Immunoblotting.
LV and liver samples were collected and stored at −80° C. for quantitative RT-PCR and Western blotting. qRT-PCR. LV and liver RNA was isolated using RNeasy mini kit (Qiagen, Valencia, Calif.) and qRT-PCR conducted as previously described′ under the following conditions: 5 min at 98° C., 40 cycles of 30 s at 95° C., 30 s at 55° C., and 30 s at 72° C. RNA equivalents were normalized to simultaneously determined glyceraldehyde-3-phosphate dehydrogenase (GAPDH) mRNA levels in each sample. Primers are listed in Table 4, below. Immunoblotting was performed as described previously.³⁵ The following antibodies were used: cMLCK (Abgen/Thermo Scientific, San Diego, Calif./Waltham Mass.); p286 CamKII (Santa Cruz, Dallas, Tex.); phospho-PKA catalytic subunit, PKA catalytic subunit, troponin I, and 22/23-phospho-troponin I (Cell Signaling Technology, Danvers Mass.); PLB (Thermo Fisher Scientific, Waltham Mass.); Ser 16 and Thr 17-phospho-PLB (Badrilla, Ltd, Leeds, UK); SERCA2a (Enzo Life Sciences, Farmingdale N.Y.).

### Cyclic AMP and Protein Kinase A (PKA) Activity.

Transmural LV samples underwent cAMP measurement before and after stimulation with isoproterenol (10 mM, 10 min) and NKH477 (10 mM, 10 min) and cAMP was measured using the Biotrak Enzyme-immunoassay System (GE Healthcare) as previously described.³⁶ PKA activity was determined as previously described.²⁷ Cardiac myocytes underwent cAMP measurement before and after stimulation with isoproterenol (10 μM, 10 min) and NKH477 (10 μM, 10 min) and subsequently homogenized in buffer A: 20 mM Tris-HCl (pH 7.4), 0.5 mM EGTA, 0.5 mM EDTA, and protease inhibitor cocktail (Invitrogen, CA) and centrifuged (14,000×g, 5 min, 4° C.). The supernatant was incubated with PKA biotinylated peptide substrate (SignaTECT®) cAMP-Dependent Protein Kinase Assay System, Promega, Madison, Wis.) in the presence of [γ-³²P]ATP. The ³²P-labeled biotinylated substrate was recovered with a streptavidin matrix and the specific activity of PKA determined.

Histology.

Samples of liver and transmural sections of the uninfarcted LV septum were formalin-fixed and paraffin-imbedded. Five micron sections were mounted and counterstained with hematoxylin and eosin and with Masson's trichrome. For quantitative assessment of LV fibrosis images of a short-axis mid-wall LV ring was obtained with a Nikon Eclipse Ti-U microscope. Blinded analysis of the degree of fibrosis in the viable LV region (excluding the infarcted region) was conducted using NIS-Elements AR 3.10 software (Nikon Inc.). A similar analytical process was performed on fixed and counterstained liver samples.

Statistical Analysis.

Data represent mean±SE; group differences were tested for statistical significance with ANOVA followed by Bonferroni t testing. Between group comparisons were made using Student's t-test (unpaired, 2-tailed). The null hypothesis was rejected when p<0.05.

Results

Heart Rate and Blood Pressure in Unsedated Mice.

No group differences were seen in heart rate or systolic, diastolic or mean arterial blood pressure 5 weeks after UCn2 gene transfer (Table 1, below), although heart rates tended to be quite high in the untreated group and closer to normal in mice that had received UCn2 gene transfer. Urocortin 2 Expression. Five weeks after intravenous delivery of AAV.UCn2 (5×10¹¹ gc; n=6), UCn2 mRNA was increased 15,263-fold in liver (p<0.0001) and 70-fold in LV (p=0.03) vs endogenous UCn2 mRNA.

Echocardiography

(Table 2, below). Intravenous delivery of AAV8.UCn2 to mice with HF was associated with increased ejection fraction (p=0.01), and velocity of circumferential fiber shortening was increased but did not reach statistical significance (p=0.09). Mice that received AAV8.UCn2 also exhibited reductions in LV end-diastolic diameter (EDD; p<0.001) and LV end-systolic diameter (ESD; p=0.002). The saline-treated mice showed an 11% increase in LV EDD, while the UCn2-treated group showed a 2% decrease in LV EDD. Similarly, the saline group showed a 16% increase in LV ESD, while the UCn2 group experienced a 6% reduction. Although these changes in LV dimension may seem small, since volume is a cubic function of dimension, the volume changes are considerable—a calculated 64% increase in ESD (saline vs UCn2) and a 46% increase in EDD (saline vs UCn2). Posterior and septal wall thickness showed no group differences (Table 1, below).

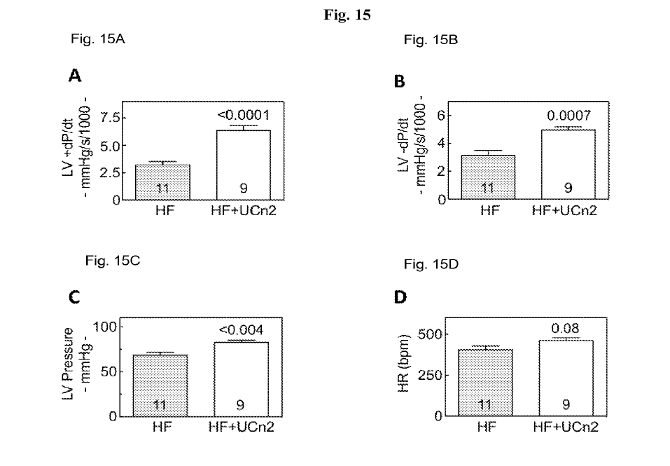

LV Systolic and Diastolic Function (FIG. 15 and Table 3, below). In vivo assessment of LV pressure development showed substantial increases in rates of LV pressure development (LV +dP/dt; p<0.0001) and in LV relaxation (LV −dP/dt; p<0.0007) (FIG. 15 and Table 3, below). There were no group differences in mean arterial pressure (Table 3). Heart rate during these studies, conducted under anesthesia, was somewhat higher in mice that had received UCn2 gene transfer, but the difference did not reach statistical significance.

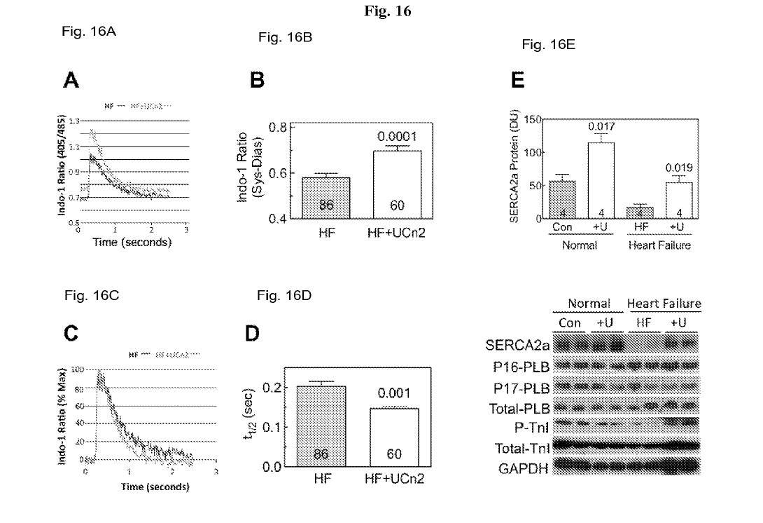

Cytosolic Ca²⁺ Transients and Related Genes. Basal Ca²⁺ released (systolic-diastolic Ca²⁺) was increased in cardiac myocytes from heart failure mice that had received UCn2 gene transfer (p=0.0001, FIGS. 16A and 16B). UCn2 gene transfer was also associated with a reduced Ca²⁺ decline time (t Tau) in cardiac myocytes from mice with heart failure 5 weeks after UCn2 gene transfer p=0.001, FIGS. 16C and 16D). Increased UCn2 was associated with increased expression of SERCA2a mRNA and protein in normal and failing LV (FIGS. 16E and 16F). However, no group difference were seen in LV protein expression and phosphorylation of phospholamban or TnI (data not shown)

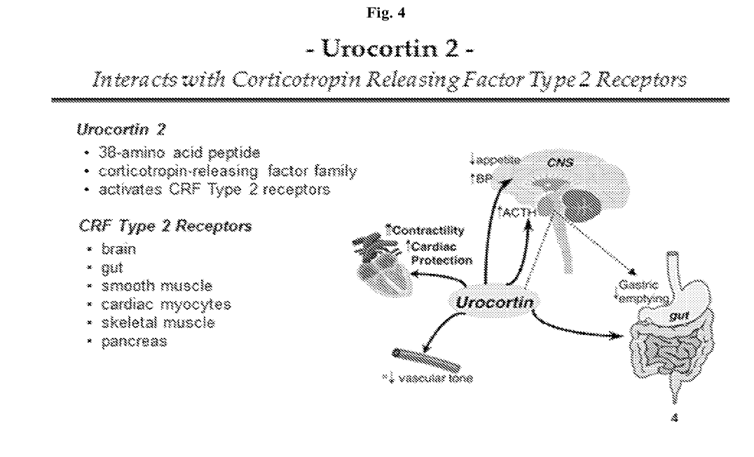

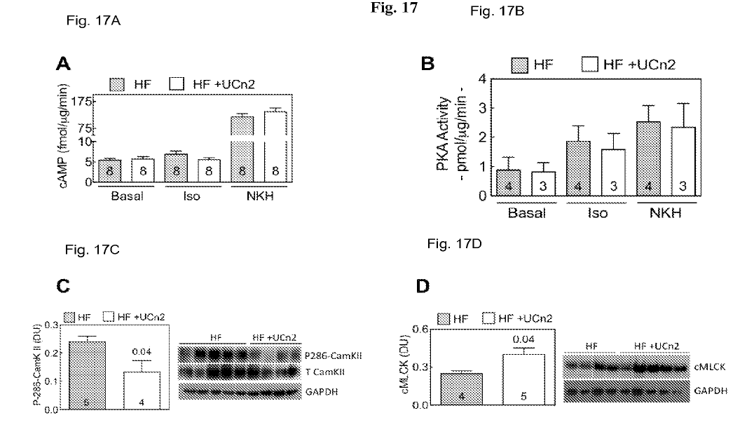

Cyclic AMP & PKA Activity. LV samples and cardiac myocytes isolated from hearts of both groups showed no differences in cAMP or PKA activity (FIG. 17). Cyclic AMP production and PKA activity were assessed before and after stimulation with isoproterenol or NKH477, a water-soluble forskolin analog that stimulates adenylate cyclase independently of β-adrenergic receptors. No group differences were seen in basal, Iso or NKH477-stimulated cAMP production (FIG. 4A) or in PKA activity (FIG. 17B). Expression of PKA family proteins (catalytic a unit and regulatory α and β subunits and their phosphorylation) was not altered (data not shown).

CamKII & cMLCK. To seek mechanisms to explain increased function of the failing heart evoked by UCn2 gene transfer, we measured LV expression and phosphorylation of calcium/calmodulin-dependent protein Kinase II (CamKII) and expression of cardiac myosin light chain kinase 3 (cMLCK). CamKII phosphorylation at Ser286 was reduced in LV samples from HF mice after UCn2 gene transfer (47% reduction, p=0.04; FIG. 4C), although total CamKII protein expression showed no group difference. Seeking alterations in myofilament sensitivity to Ca²⁺ we assessed LV cardiac myosin light chain kinase 3 (cMLCK) expression after UCn2 gene transfer, finding a 1.6-fold increase (p<0.04) (FIG. 4D).

Necropsy (Table 5). Liver, lung and body weights showed no group differences. UCn2 gene transfer tended to reduce LV weight, and LV to body weight ratio was reduced (12% reduction. p=0.01).

Markers of Stress, Inflammation and Tissue Injury (Table 6, below). The expression of several markers of LV stress, inflammation and tissue injury was examined using RT-PCR. HF altered the expression of most of these genes (Table 6). Increased UCn2 expression did not influence alterations associated with HF. However, in normal mice, increased UCn2 expression was associated with reduced expression of ANF (p=0.007), BNP (p=0.01), β-MyHC and α-SK-actin (p=0.03).

LV and Liver Histology. Hematoxylin and eosin staining of samples of liver and LV showed no evidence of group differences (data not shown). Masson's trichrome staining revealed no group differences in fibrosis in liver (p=0.79).

### DISCUSSION

This study demonstrated that a single intravenous injection of AAV8.UCn2 increased function of the failing heart, demonstrating the feasibility and effectiveness of intravenous delivery of a long term expression vector encoding a peptide with beneficial paracrine effects to treat heart failure.

Two measures of cardiac function confirmed increased LV function 5 weeks after IV AAV8.UCn2 delivery to animals with severely dysfunctional left ventricles. Echocardiography showed increases in LV ejection fraction, and reductions in LV volumes (Table 1). Secondly, UCn2 gene transfer increased peak LV +dP/dt, indicating enhanced LV contractile function, and reduced LV −dP/dt, indicating enhanced LV diastolic function (Table 3, FIG. 15).

Although the absolute degree of LV EF change was only 8 percentage units (HF: 12±1%; HF+UCn2: 20±4%), the relative increase was 67%. The small absolute change reflects the large size of the infarction—the mean pre-randomization LV EFs were <20% in both groups. Despite such large infarctions, UCn2 gene transfer attenuated LV chamber dilation and increased EF, while saline-treated mice showed progressive LV chamber dilation and further deterioration of LV EF. One would not expect UCn2 gene transfer to remedy the problems associated with such a large area of scar, representing virtually the entirety of the LV free wall. The cardiac benefits of UCn2 gene transfer would be anticipated to be limited to the viable portion of the LV, which, in the current model, represents the interventricular septum. Ejection fraction in this setting may underestimate the benefits on LV function, especially since we observed dyskinesia of the infarcted wall during ejection. Assessment of LV contractile function using peak LV +dP/dt reveals a larger absolute increase in LV function—an increase of 3129 mmHg/sec in peak +LV dP/dt, and a 1857 mmHg/sec increase in peak −dP/dt conferred by UCn2 gene transfer. These represent a 2-fold increase in peak +LV dP/dt, and a 1.6 fold increase in peak −dP/dt. A doubling of peak LV +dP/dt in clinical heart failure would normalize LV contractile function.^{37,38}

Heart rate and blood pressure in the unsedated state are not affected by intravenous delivery of AAV8.UCn2 despite sustained high levels of transgene UCn2 in normal mice (27) or in mice with CHF, as shown in the current study. Similarly, in clinical trials of peptide infusions of UCn2 and stresscopin (similar to UCn3) the rate-pressure product is unchanged (9-11). One would, therefore, not anticipate an increase in cardiac metabolic demands associated with UCn2 gene transfer, but more direct metabolic studies must be performed to know this with certainty.

The present study focused on the feasibility and physiological consequences of intravenous delivery of AAV8.UCn2 in the setting of a severely compromised and failing heart, and we found a pronounced positive effect. The mechanisms by which UCn2 gene transfer evoked beneficial physiological changes, although not the primary focus of the present study, were also examined.

For example, we found that UCn2 gene transfer was associated with a) increased peak systolic Ca²⁺ transient amplitude and increased rate of Ca²⁺ decline in cardiac myocytes isolated from HF mice (FIG. 16A-16D); and b) increased SERCA2a expression (FIGS. 16E and 16F) as we previously reported in mice with normal hearts.²⁷ Increased LV SERCA2a expression provides a mechanism by which LV contractile function and relaxation would be increased, as was observed (FIG. 15). SERCA2a returns cytosolic Ca²⁺ to the sarcoplasmic reticulum. An increased amount of SERCA2a would be anticipated to yield a more rapid cytosolic Ca²⁺ decline, which is what we found (FIGS. 16C and 16D), and consequently to increase the rate of LV pressure decline (LV −dP/dt), as we also found (FIG. 15B).

In addition, we found alterations in LV expression of two additional proteins that are likely to have been of mechanistic importance in the observed beneficial effects of UCn2 gene transfer on function of the failing LV: reduced Thr286 phosphorylation of Ca²⁺/calmodulin-dependent kinase II (CaMKII), and increased LV expression of cardiac myosin light chain kinase (cMLCK) (FIG. 4). CaMKII Thr286 Phosphorylation. Our data show that UCn2 gene transfer was associated with reduced Thr286 phosphorylation of CaMKII (FIG. 17C). CaMKII expression and activation are important determinants of cardiac function.³⁹ For example, cardiac-directed expression of CaMKII results in heart failure in mice.⁴⁰ Others have shown increased CaMKII activity and expression in MI-induced heart failure in mice.⁴¹ The clinical relevance of these findings was demonstrated recently by the demonstration that inhibition of LV CaMKII increases function of the failing human heart.⁴² Although we speculate that reduced Thr286 phosphorylation of CaMKII may have been important mechanistically in the observed increase in LV function, we were unable to determine the pathway by which increased UCn2 reduces Thr286 CaMKII phosphorylation, which will require focused studies in cultured cardiac myocytes that are underway. Cardiac Myosin Light Chain Kinase (cMLCK). We found increased cMLCK expression associated with UCn2 gene transfer (FIG. 17D). Phosphorylation of cardiac myosin light chain 2 v by cMLCK increases the rate of cross-bridge recruitment in cardiac myocytes and influences contractile function.^{43,44} Increased levels cMLCK are associated with increased LV function in the setting of MI-induced heart failure.⁴⁵ In contrast, the deletion of cMLCK reduces cardiac performance.⁴⁶ Sadly, there is no antibody available to assess myosin light chain 2 v phosphorylation, so the biological importance of the increase in cMLCK associated with UCn2 gene transfer in the present study must remain speculative.

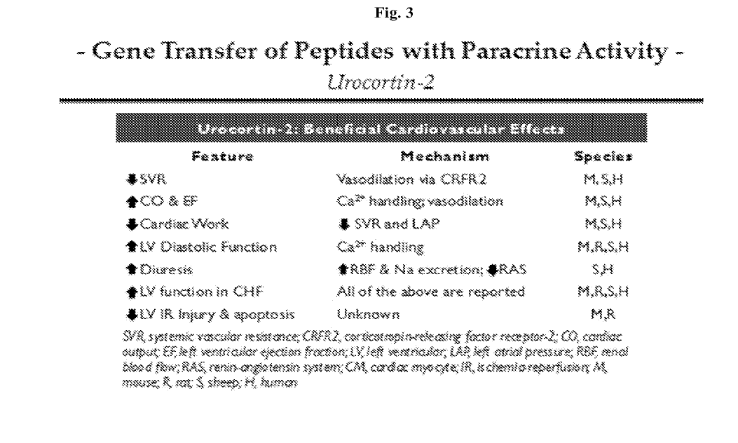

UCn2 gene transfer was associated with a doubling in the peak rate of LV pressure development (LV +dP/dt; Table 3 and FIG. 15). This finding was supported by evaluation of LV dimension and function by echocardiography (Table 2), enhanced Ca²⁺ handling (FIG. 3), and signaling changes in LV predicted to increase contractile function, including increased SERCA2a protein expression (FIG. 16 and FIG. 17). Because of the consistency of these findings, which reverberated from isolated cardiac myocytes to in vivo physiology, we were less concerned by the absence of group differences in BNP and ANF mRNA in LV (Table 6). Perhaps plasma levels or BNP/ANF expression in LA would have revealed group differences that LV mRNA levels missed. It is also possible that despite increased LV contractile function there was sufficient persistent chamber dilation—owing to infarction of the entire LV free wall—to provide ongoing stimulation of ANF and BNP expression.

We saw no group difference in lung or liver weight (Table 5). Liver weights were not increased in mice with heart failure compared to normal mice (27), so, despite severe left ventricular (LV) failure, there is no liver congestion. Whether this is unique to MI-induced CHF in mice is unknown. Lung weights increased by 23% vs normal age-matched mice (27), but did not show a group difference. We speculate that despite a doubling of LV contractile function (peak +dP/dt) conferred by UCn2 gene transfer (Table 3 and FIG. 15), there may have been persistent left sided congestion 5 weeks after treatment.

Clinical Application. Intravenous delivery of AAV8 enables transfection of many organs and is especially effective in liver, skeletal muscle and heart.⁴⁸ These organs, because they comprise an enormous mass of tissue and therefore can release abundant transgene UCn2, will enable us to reduce the vector dose. Indeed, a vector dose 10-fold lower (5×10¹⁰ gc per mouse or 2×10¹² gc/kg) is still effective in increasing LV +dP/dt (27). A dose of 2×10¹² gc/kg of AAV8 encoding human Factor IX was delivered intravenously safely and effectively in a clinical trial in subjects with hemophilia B.²

An additional feature to consider in translating our findings to clinical applications is the use of a regulated expression system,^{5,6,9} which would enable turning UCn2 expression on or off at will. We have designed such AAV8 vectors using tetracycline and rapamycin regulation systems and are conducting preclinical studies with these regulated expression vectors.

LV Ca²⁺ handling is different in humans than in mice,⁴⁷ but peptide infusions of UCn2 or stresscopin (similar to UCn3) in patients with HF increases LV function (9-11). Whether this is through Ca²⁺ handling is unknown because Ca²⁺ transients and Ca²⁺ handling proteins have not been assessed in cardiac myocytes or myocardium before and after UCn2 peptide infusions in humans.

Finally, now that we have demonstrated that UCn2 gene transfer increases function of the severely failing heart, it will be important to determine how long the effect persists and whether it reduces mortality. Such studies using a less severe model of CHF with better long-term survival are planned.

These data demonstrate that a single intravenous injection of AAV8.UCn2 increases both systolic and diastolic function of the severely failing heart. Systemic delivery of the vector ensures that the transgene is expressed in the heart, but also is continuously released into the circulation, thereby providing sustained benefits that would otherwise not be possible. Other advantages of gene transfer as compared to IV infusion of paracrine acting peptides include reduction in catheter-based infections, no need for hospitalization, and reduced costs.

### FIGURE LEGENDS

### Example 2

### FIG. 14. AAV8.CBA.UCn2 Map and Experimental Protocol

A. AAV8.CBA.UCn2 Vector Map: ITR, inverted terminal repeat; SVpA, polyA from SV40 viral genome; UCn2, urocortin-2; CBA, chicken β-actin promoter; CMV. en, human cytomegalovirus enhancer
B. Experimental Protocol. Normal mice underwent myocardial infarction (MI, by proximal left coronary ligation) to induce HF, which was assessed by echocardiography 3 weeks after MI. Mice with EF<25% were then randomized to receive AAV8.UCn2 (5×10¹¹ gc, IV) or IV saline. Five weeks later echocardiography was used to assess LV size and function. In vivo physiological studies were conducted to evaluate rates of LV pressure development (LV +dP/dt) and decay (LV −dP/dt), to assess LV systolic and diastolic function. Cross sections of LV (mid-papillary level) show that the infarction is extensive, comprising the majority of the LV free wall, with only the interventricular septum spared. Data acquisition and analysis were blinded to group treatment.

### FIG. 15. LV Function In Vivo

A and B. Five weeks after AAV8.UCn2 (5×10¹¹ gc, IV) or saline (HF) in vivo studies were performed to measure the rate of LV pressure development (LV +dP/dt; A) and decay (LV −dP/dt; B). AAV8.UCn2 increased LV +dP/dt and LV −dP/dt 5 weeks after gene transfer, indicating that UCn2 gene transfer increase LV systolic function.

C and D. Heart rate tended to be higher (D). LV developed pressure was increased by UCn2 gene transfer (C). Studies were performed without knowledge of group identity.

P values are from Student's t-test (unpaired, two-tailed). Data represent mean±SE, and numbers in bars denote group size.

### FIG. 16. Cytosolic Ca²⁺ Transients in Cardiac Myocytes from Mice with Heart Failure (HF)

5 w after IV AAV8.UCn2 (HF+UCn2) or IV saline. A and B. Basal Ca²⁺ released (systolic-diastolic CO was increased in cardiac myocytes from HF+UCn2 mice (p=0.0001). A. Representative Indo-1 Ca²⁺ transient recordings from one heart in each group showed increased peak Ca²⁺ in cardiac myocytes isolated from mice with heart failure 5 weeks after UCn2 gene transfer. B. Summary data from 3 mice per group are shown. C and D. Time to Ca²⁺ decline (t Tau) was shortened in cardiac myocytes from mice with heart failure 5 weeks after UCn2 gene transfer.

C. Representative normalized Ca²⁺ transients from cardiac myocytes from one heart in each group. D. Summary data from 3 mice per group are shown. For A and C, each curve is the average of 30 cardiac myocytes from one heart from each group. For B and D, summary data from 3 animals per group include analysis of 144 individual cardiac myocytes (86, saline; 60, AAV8.UCn2). For B and D, bars denote mean+SE; numbers in bars denote number of cardiac myocytes; numbers above bars indicate p values from Student's t-test (unpaired, 2-tailed).

E. Summary (top panel) of immunoblotting data (bottom panel) indicates that UCn2 gene transfer increased SERCA2a protein in LV from normal mice and from mice with heart failure. Expression and phosphorylation of phospholamban (PLB) and troponin I (TnI) were not affected. Bars denote mean+SE; numbers in bars denote group size; numbers above bars from Student's t-test (unpaired, 2 tails vs control).

### FIG. 17. Cardiac Myocyte cAMP-PKA Signaling

LV samples (A, C, D) or cardiac myocytes (B) were obtained from mice with heart failure (HF) and from mice with HF that had received AAV8.UCn2 (UCn2). Cyclic AMP and PKA activity were assessed in the unstimulated (basal) state and after stimulation with isoproterenol (Iso, 10 μM, 10 min) and, in separate experiments, NKH477 (NKH, 10 μM, 10 min), a water-soluble forskolin analog that stimulates adenylate cyclase independent of β-adrenergic receptors. Numbers in bars denote group size.
A. cAMP Production: No group differences were seen in basal, Iso or NKH477-stimulated cAMP production.
B. PKA Activity: No group differences were seen in basal, Iso or NKH477-stimulated conditions.
C. CamK II Expression and Phosphorylation: UCn2 gene transfer was associated with reduced Thr286 phosphorylation of CamK II (Left panel, normalized to GAPDH). Total CamK II was unchanged.
D. Cardiac Myosin Light Chain Kinase: UCn2 gene transfer was associated with increased cardiac myosin light chain kinase (cMLCK) protein (Left panel, normalized to GAPDH).

In all graphs, bars denote mean+SE; numbers in bars denote group size, numbers above bars from Student's t-test (unpaired, 2 tails vs control groups).

TABLE 1
Effects of UCn2 Gene Transfer on Heart Rate & Blood Pressure in
Mice with Heart Failure

| | | HF + UCn2 | |
|---|---|---|---|
| | HF (n) | (n) | p |
| Heart Rate | 693 ± 54 (4) | 601 ± 96 (5) | 0.13 |
| beats/min | | | |
| Systolic Pressure | 123 ± 23 (5) | 105 ± 17 (5) | 0.20 |
| mmHg | | | |
| Diastolic Pressure | 89 ± 18 (5) | 73 ± 14 (5) | 0.16 |
| mmHg | | | |
| Mean Arterial | 100 ± 19 (5) | 83 ± 16 (5) | 0.28 |
| Pressure | | | |
| mmHg- | | | |

The effects of UCn2 gene transfer on blood pressure and heart rate (HR) were assessed in unsedated mice with heart failure (HF) 5 weeks after UCn2 gene transfer (HF+UCn2, 5×10¹¹ gc, IV) or IV saline (HF). Systolic and diastolic blood pressure

TABLE 2
Echocardiography Before and After UCn2 Gene Transfer vs Saline for HF

| | HF (12) | | | HF + UCn2 (13) | | | |
|---|---|---|---|---|---|---|---|
| | 3 Weeks | 5 Weeks | | 3 Weeks | 5 Weeks | | |
| | after MI | after Saline | Post-Pre | after MI | after UCn2 | Post-Pre | p |
| HR (bpm) | 542 ± 18 | 513 ± 13 | −29 ± 21 | 503 ± 12 | 525 ± 12 | 22 ± 13 | 0.045 |
| EDD (mm) | 5.3 ± 0.3 | 5.9 ± 0.3 | 0.6 ± 0.1 | 5.3 ± 0.3 | 5.2 ± 0.3 | −0.1 ± 0.1 | <0.001 |
| ESD (mm) | 4.5 ± 0.4 | 5.2 ± 0.4 | 0.7 ± 0.2 | 4.7 ± 0.4 | 4.4 ± 0.4 | −0.3 ± 0.2 | 0.002 |
| LVEF (%) | 19 ± 2 | 12 ± 1 | −7 ± 2 | 17 ± 2 | 20 ± 4 | 3 ± 3 | 0.01 |
| V_{CFc} (circ/sec) | 3.3 ± 0.9 | 3.0 ± 0.8 | −0.3 ± 0.6 | 3.5 ± 0.7 | 4.7 ± 0.8 | 1.2 ± 0.6 | 0.09 |
| PW Th (mm) | 0.5 ± 0.03 | 0.5 ± 0.03 | −0.05 ± 0.03 | 0.5 ± 0.03 | 0.5 ± 0.03 | −0.01 ± 0.02 | 0.20 |
| IVS Th (mm) | 0.5 ± 0.04 | 0.5 ± 0.04 | 0.01 ± 0.02 | 0.5 ± 0.04 | 0.5 ± 0.05 | −0.02 ± 0.04 | 0.43 |

HF, heart failure; UCn2, urocortin-2; HR, heart rate; bpm, beats per minute; EDD, LV end-diastolic diameter; ESD, LV end-systolic diameter; LVEF, left ventricular ejection; VCFc, velocity of circumferential fiber shortening (corrected for heart rate); PW Th, posterior wall thickness at end-diastole; IVS Th, interventricular wall thickness at end-diastole; Post-Pre, the value 5 weeks after Saline or UCn2 gene transfer minus the value before. P values from Student's t-test (paired data, 2 tails) for group difference in change, Post-Pre.
was measured by tail cuff and mean blood pressure calculated. No group differences were seen in heart rate or blood pressure. Values denote mean±SE; p values are from Student's t-test (unpaired, two-tailed).

TABLE 3
Physiological Data

| | Saline (11) |
|---|---|
| LVP (mmHg) | 68 ± 3 |
| LV +dP/dt (mmHg/s) | 3225 ± 287 |
| LV −dP/dt (mmHg/s) | −3127 ± 370 |
| MAP (mmHg) | 56 ± 3 |
| HR (bpm) | 404 ± 23 |

Three weeks after myocardial infarction, mice received intravenous saline or AAV8.UCn2 (5×10¹¹ gc). Mice underwent physiological studies 5 weeks later. LVP, left ventricular developed pressure; LV, left ventricle; MAP, mean arterial pressure; HR, heart rate; UCn2, Urocortin-2 gene transfer. Values represent mean±SE. P values are from Student's t-test (unpaired, two-tailed).

TABLE 4
Primers

| Gene | Forward | Reverse |
|---|---|---|
| ANF | 5′-CCTCGTCTTGGCCTTTTGG | 5′-CATCTTCTACCGGCATCTTC |
| | (SEQ ID NO: 1) | (SEQ ID NO: 2) |
| α-MHC | 5′-AAAGGCTGAGAGGAACTACC | 5′-ACCAGCCTTCTCCTCTGC |
| | (SEQ ID NO: 3) | (SEQ ID NO: 4) |
| α-Cd-actin | 5′-GTGTTACGTCGCCCTTGATT | 5′-TGAAAGAGGGCTGGAAGAGA |
| | (SEQ ID NO: 5) | (SEQ ID NO: 6) |
| α-SK-actin | 5′-GTGTCACCCACAACGTGC | 5′-AGGGCCACATAGCACAGC |
| | (SEQ ID NO: 7) | (SEQ ID NO: 8) |
| β-MHC | 5′-GCTGAAAGCAGAAAGAGATTATC | 5′-TGGAGTTCTTCTCTTCTGGAG |
| | (SEQ ID NO: 9) | (SEQ ID NO: 10) |
| BNP | 5′-GAAGTCCTAGCCAGTCTCC | 5′-CAGCTTGAGATATGTGTCACC |
| | (SEQ ID NO: 11) | (SEQ ID NO: 12) |
| Coll1αl | 5′-GCCAAGAAGACATCCCTGAAG | 5′-GGGTCCCTCGACTCCTAC |
| | (SEQ ID NO: 13) | (SEQ ID NO: 14) |
| Coll3α1 | 5′-GCACAGCAGTCCAACGTAGA | 5′-TCTCCAAATGGGATCTCTGG |
| | (SEQ ID NO: 15) | (SEQ ID NO: 16) |
| GAPDH | 5′-CATGTTCCAGTATGACTCCACTC | 5′-GGCCTCACCCCATTTGATGT |
| | (SEQ ID NO: 17) | (SEQ ID NO: 18) |
| MEF2 | 5′-GAGCCTCATGAAAGCAGGAC | 5′-GAAGTTCTGAGGTGGCAAGC |
| | (SEQ ID NO: 19) | (SEQ ID NO: 20) |
| MMP2 | 5′-GAGTTGCAACCTCTTTGTGC | 5′-CAGGTGTGTAACCAATGATCC |
| | (SEQ ID NO: 21) | (SEQ ID NO: 22) |
| MMP8 | 5′-GACTCTGGTGATTTCTTGCTAAC | 5′-CACCATGGTCTCTTGAGACG |
| | (SEQ ID NO: 23) | (SEQ ID NO: 24) |
| MMP9 | 5′-CGTCGTGATCCCCACTTACT | 5′-GAACACACAGGGTTTGCCTTC |
| | (SEQ ID NO: 25) | (SEQ ID NO: 26) |
| TIMP1 | 5′-GACAGCTTTCTGCAACTCGG | 5′-CTTGTGGACATATCCACAGAGG |
| | (SEQ ID NO: 27) | (SEQ ID NO: 28) |
| TIMP2 | 5′-GCAATGCAGACGTAGTGATCAG | 5′-CCTTCTTTCCTCCAACGTCC |
| | (SEQ ID NO: 29) | (SEQ ID NO: 30) |
| TIMP3 | 5′-CTTCTGCAACTCCGACATCG | 5′-CCTGTCAGCAGGTACTGG |
| | (SEQ ID NO: 31) | (SEQ ID NO: 32) |
| TIMP4 | 5′-CAAGGATATTCAGTATGTCTACACG | 5′-CTGGTGGTAGTGATGATTCAGG |
| | (SEQ ID NO: 33) | (SEQ ID NO: 34) |
| UCn2 | 5′-ACTCCTATCCCCACCTTCCA | 5′-AAGATCCGTAGGAGGCCAAT |
| | (SEQ ID NO: 35) | (SEQ ID NO: 36) |

ANF, atrial natriuretic peptide; α-MHC, alpha-myosin heavy chain; α-Cd-Actin, alpha-cardiac actin; α-SK-Actin, alpha-skeletal actin; β-MHC, beta-myosin heavy chain; BNP, brain natriuretic peptide; Coll, collagen; MMP, matrix metallopeptidase; TIMP, tissue inhibitor of metalloproteinases; MEF2, myocyte enhancer factor-2; UCn2, urocortin 2.

TABLE 5
Necropsy

| Saline (17) | UCn2 (17) |
|---|---|
| 30 ± 1 | 31 ± 1 |
| 154 ± 7 | 139 ± 5 |
| 5.1 ± 0.2 | 4.5 ± 0.1 |
| 1489 ± 53 | 1405 ± 43 |
| 212 ± 19 | 213 ± 13 |

Three weeks after myocardial infarction, mice received intravenous saline or AAV8.UCn2 (5×10¹¹ gc). Mice were killed 6 weeks later and necropsy conducted. BW, body weight; g, grams; LV, left ventricle; UCn2, Urocortin-2 gene transfer. Values represent mean±SE. P values are from Student's t-test (unpaired, two-tailed).

TABLE 6
mRNA Expression in Left Ventricle

| | Normal | | HF | | | UCn2 | HF |
|---|---|---|---|---|---|---|---|
| Gene | Control | UCn2 | Control | UCn2 | Interaction | Effect | Effect |
| ANF | 100 ± 17 | 38 ± 7 | 2393 ± 591 | 2458 ± 728 | ns | ns | 0.0001 |
| α-MHC | 100 ± 10 | 83 ± 26 | 837 ± 90 | 714 ± 76 | ns | ns | 0.0001 |
| α-Cd-Actin | 100 ± 7 | 164 ± 70 | 1160 ± 94 | 1368 ± 134 | ns | ns | 0.0001 |
| α-sk-Actin | 100 ± 32 | 18 ± 4 | 56 ± 12 | 51 ± 14 | 0.05 | 0.03 | ns |
| β-MHC | 100 ± 33 | 11 ± 3 | 104 ± 23 | 74 ± 20 | ns | 0.016 | ns |
| BNP | 100 ± 16 | 44 ± 9 | 484 ± 098 | 525 ± 152 | ns | ns | 0.0001 |
| MMP2 | 100 ± 9.5 | 102 ± 14 | 707 ± 304 | 601 ± 41 | ns | ns | 0.002 |
| MMP8 | 100 ± 38 | 68 ± 9.6 | 96 ± 36 | 90 ± 50 | ns | ns | ns |
| MMP9 | 100 ± 44 | 68 ± 2.3 | 57 ± 20 | 44 ± 21 | ns | ns | ns |
| TIMP1 | 100 ± 47 | 69 ± 6 | 250 ± 62 | 341 ± 49 | ns | ns | 0.0002 |
| TIMP2 | 100 ± 7 | 122 ± 16 | 500 ± 65 | 719 ± 106 | ns | ns | 0.0001 |
| TIMP3 | 100 ± 13 | 52 ± 4 | 207 ± 42 | 269 ± 43 | ns | ns | 0.0001 |
| TIMP4 | 100 ± 22 | 86 ± 16 | 239 ± 50 | 164 ± 21 | ns | ns | 0.002 |
| Coll1α1 | 100 ± 10 | 152 ± 7 | 183 ± 45 | 257 ± 38 | ns | ns | 0.005 |
| Coll3α1 | 100 ± 11 | 140 ± 17 | 281 ± 80 | 376 ± 62 | ns | ns | 0.0006 |
| MEF2 | 100 ± 9 | 132 ± 78 | 1486 ± 174 | 1682 ± 155 | ns | ns | 0.0001 |

### REFERENCES

### Example 1

1. Gao M H, Lai N C, Miyanohara A, Schilling J M, Suarez J, Tang T, Guo T, Tang R, Parikh J, Giamouridis D, Dillmann W H, Patel H H, Roth D M, Dalton N D, Hammond H K. Intravenous adeno-associated virus serotype 8 encoding urocortin-2 provides sustained augmentation of left ventricular function in mice. 24:777-7785, 2013
2. Robertson R P. Islet transplantation a decade later and strategies for filling a half-full glass. Diabetes 2010; 59:1285-1291
3. Callejas D, Mann C J, Ayuso E, Lage R, Grifoll I, Roca C, Andaluz A, Ruiz-de Gopegui R, Montane J, Muñoz S, Ferre T, Haurigot V, Zhou S, Ruberte J, Mingozzi F, High K A, Garcia F, Bosch F. Treatment of diabetes and long-term survival after insulin and glucokinase gene therapy. Diabetes 2013; 62: 1718-1729
4. Gaddy D F, Riedel M J, Pejawar-Gaddy S, Kieffer T J, Robbins P D. In vivo expression of HGF/NK1 and GLP-1 From dsAAV vectors enhances pancreatic B-cell proliferation and improves pathology in the db/db mouse model of diabetes. Diabetes 2010; 59:3108-3116
5. Grines C L, Watkins M W, Helmer G, Penny W, Brinker J, Marmur J D, West A, Rade J J, Marratt P, Hammond H K, Engler R L. Angiogenic gene therapy (AGENT) trial in patients with stable angina pectoris. Circulation 2002; 105:1291-1297
6. AC6 Gene Transfer for CHF, ClinicalTrials.gov NCT00787059
7. Marsh S A, Dell'Italia L J, Chatham J C. Interaction of diet and diabetes on cardiovascular function in rats. Am J Physiol Heart Circ Physiol 2009; 296: H282-H292
8. Lai N C, Tang T, Gao M H, Saito M, Takahashi T, Roth D M, Hammond H K. Activation of cardiac adenylyl cyclase expression increases function of the failing ischemic heart in mice. J Am Coll Cardiol 2008; 51: 1490-1497
9. Gao M H, Bayat H, Roth D M, Yao Zhou J, Drumm J, Burhan J, Hammond H K. Controlled expression of cardiac-directed adenylylcyclase type VI provides increased contractile function. Cardiovasc Res 2002; 56: 197-204
10. Tang T, Hammond H K, Firth A, Yang Y, Gao M H, Yuan J X J, Lai N C. Adenylyl cyclase 6 improves calcium uptake and LV function in aged heart. J Am Coll Cardiol 2011; 57: 1846-1855
11. Lai N C, Tang T, Gao M H, Saito M, Miyanohara A, Hammond H K. Improved function of the failing rat heart by regulated expression of insulin-like growth factor I via intramuscular gene transfer. Hum Gene Ther 2012; 23: 255-261
12. Gao M H, Lai N C, Miyanohara A, Suarez J, Giamouridis D, Theodore P. Ciaraldi T P, Schenk S, Hammond H K. Intravenous adeno-associated virus serotype 8 encoding urocortin-2 normalizes hyperglycemia in type-2 diabetes mellitus in mice (Submitted)
13. Gao M H, Lai N C, Giamouridis D, Miyanohara A, Suarez J, Parikh J, Hightower S, Guo T, Dillmann W H, Hammond H K. Intravenous AAV8 encoding urocortin 2 increases function of the failing heart in mice (Submitted)
14. Perrin M H, Vale W W. Corticotropin releasing factor receptors and their ligand family. Ann N Y Acad Sci. 1999; 885:312-328. PMID: 10816663
15. Davis M E, Pemberton C J, Yandle T G et al. Urocortin 2 infusion in human heart failure. Eur Heart J 2007; 28: 2589-2597
16. Gheorghiade M, Greene S J, Ponikowski P, Maggioni A P, Korewicki J, Macarie C, Metra M, Grzybowski J, Bubenek-Turconi S I, Radziszewski W, Olson A, Bueno O F, Ghosh A, Deckelbaum L I, Li L Y, Patel A R, Koester A, Konstam M A. Haemodynamic effects, safety, and pharmacokinetics of human stresscopin in heart failure with reduced ejection fraction. Eur J Heart Fail 2013; 6: 679-689.
17. Nathwani A C, Tuddenham E G, Rangarajan S et al. Adenovirus-associated virus vector-mediated gene transfer in hemophilia B. N Engl J Med 2011; 365: 2357-2365
18. Flotte T R, Trapnell B C, Humphries M, Carey B, Calcedo R, Rouhani F, Campbell-Thompson M, Yachnis A T, Sandhaus R A, McElvaney N G, Mueller C, Messina L M, Wilson J M, Brantly M, Knop D R, Ye G J, Chulay J D. Phase 2 clinical trial of a recombinant adeno-associated viral vector expressing alphal-antitrypsin: interim results. Hum Gene Ther 2011; 22: 1239-1247. PMCID: PMC3205788
19. Nathwani A C, Rosales C, McIntosh J, Rastegarlari G, Nathwani D, Raj D (and 18 others). Long-term safety and efficacy following systemic administration of a self-complementary AAV vector encoding human FIX pseudotyped with serotype 5 and 8 capsid proteins. Mol Ther 2011; 19: 876-885. PMCID: PMC3098629
20. Rivera V M, Gao G P, Grant R L, Schnell M A, Zoltick P W, Rozamus L W Clackson T, Wilson J M. Long-term pharmacologically regulated expression of erythropoietin in primates following AAV-mediated gene transfer. Blood 2005; 105: 1424-1430. PMID: 15507527
21. Lai N C, Tang T, Gao M H, Saito M, Miyanohara A, Hammond H K. Improved function of the failing rat heart by regulated expression of insulin-like growth factor I via intramuscular gene transfer. Hum Gene Ther 2012; 23: 255-261. PMID: 22017392
22. Mingozzi F, Meulenberg J J, Hui D J, Basner-Tschakarjan E, Hasbrouck N C, Edmonson S A, Hutnick N A, Betts M R, Kastelein J J, Stroes E S, High K A. AAV-1-mediated gene transfer to skeletal muscle in humans results in dose-dependent activation of capsid-specific T cells. Blood 2009; 114: 2077-2086. PMCID: PMC2744569
23. Hildinger M, Auricchio A, Gao G, Wang L, Chirmule N, Wilson J M. Hybrid vectors based on adeno-associated virus serotypes 2 and 5 for muscle-directed gene transfer. J Virol 2001; 75: 6199-6203. PMCID: PMC114336
24. Fang H, Lai N C, Gao M H, Miyanohara A, Roth D M, Tang T, Hammond H K. Comparison of adeno-associated virus serotypes and delivery methods for cardiac gene transfer. Hum Gene Ther Methods 2012; 23: 234-241. PMID: 22966786
25. Boutin S, Monteilhet V, Veron P, Leborgne C, Benveniste O, Montus M F, Masurier C. Prevalence of serum IgG and neutralizing factors against adeno-associated virus (AAV) types 1, 2, 5, 6, 8, and 9 in the healthy population: implications for gene therapy using AAV vectors. Hum Gene Ther 21:704-712, 2010
26. Everett R S, Evans H K, Hodges B L, Ding E Y, Serra D M, Amalfitano A. Strain-specific rate of shutdown of CMV enhancer activity in murine liver confirmed by use of persistent [E1(−), E2b(−)] adenoviral vectors. Virology 2004; 325: 96-105. PMID: 15231389
27. Stieger K, Belbellaa B, Le Guiner C, Moullier P, Rolling F. In vivo gene regulation using tetracycline-regulatable systems. Adv Drug Deliv Rev 2009; 61: 527-541. PMID: 19394373
28. Grines C L, Watkins M W, Helmer G, Penny W, Brinker J, Marmur J D, West A, Rade J J, Marratt P, Hammond H K, Engler R L. Angiogenic gene therapy (AGENT) trial in patients with stable angina pectoris. Circulation 2002; 105:1291-1297
29. AC6 Gene Transfer for CHF, ClinicalTrials.gov NCT00787059
30. Lai N C, Tang T, Gao M H, Saito M, Takahashi T, Roth D M, Hammond H K. Activation of cardiac adenylyl cyclase expression increases function of the failing ischemic heart in mice. J Am Coll Cardiol 2008; 51: 1490-1497
31. Gao M H, Bayat H, Roth D M, Yao Zhou J, Drumm J, Burhan J, Hammond H K. Controlled expression of cardiac-directed adenylylcyclase type VI provides increased contractile function. Cardiovasc Res 2002; 56: 197-204
32. Tang T, Hammond H K, Firth A, Yang Y, Gao M H, Yuan J X J, Lai N C. Adenylyl cyclase 6 improves calcium uptake and LV function in aged heart. J Am Coll Cardiol 2011; 57: 1846-1855
33. Lai N C, Tang T, Gao M H, Saito M, Miyanohara A, Hammond H K. Improved function of the failing rat heart by regulated expression of insulin-like growth factor I via intramuscular gene transfer. Hum Gene Ther 2012; 23: 255-261
34. Marsh S A, Dell'Italia L J, Chatham J C. Interaction of diet and diabetes on cardiovascular function in rats. Am J Physiol Heart Circ Physiol 2009; 296: H282-H292
35. Nemoto O, Kawaguchi M, Yaoita H, Miyake K, Maehara K, Maruyama Y. Left ventricular dysfunction and remodeling in streptozotocin-induced diabetic rats. Circ J 2006; 70: 327-334
36. Robertson R P. Islet transplantation a decade later and strategies for filling a half-full glass. Diabetes 2010; 59:1285-1291
37. Callejas D, Mann C J, Ayuso E, Lage R, Grifoll I, Roca C, Andaluz A, Ruiz-de Gopegui R, Montane J, Munoz S, Ferre T, Haurigot V, Zhou S, Ruberte J, Mingozzi F, High K A, Garcia F, Bosch F. Treatment of diabetes and long-term survival after insulin and glucokinase gene therapy. Diabetes 2013; 62: 1718-1729
38. Gaddy D F, Riedel M J, Pejawar-Gaddy S, Kieffer T J, Robbins P D. In vivo expression of HGF/NK1 and GLP-1. From dsAAV vectors enhances pancreatic B-cell proliferation and improves pathology in the db/db mouse model of diabetes. Diabetes 2010; 59: 3108-3116

### REFERENCES

### Example 2

1. Roger V. L., Go A. S., Lloyd-Jones D. M., et al. (2012). Heart disease and stroke statistics—2012 update: a report from the American Heart Association. Circulation 125, e2-e220.
2. Nathwani A. C., Tuddenham E. G. D., Rangarajan S., et al. (2011). Adenovirus-associated virus vector-mediated gene transfer in hemophilia B. N. Engl. J. Med. 365, 2357-2365.
3. Buchlis G., Podsakoff G. M., Radu A., et al. (2012). Factor IX expression in skeletal muscle of a severe hemophilia B patient 10 years after AAV-mediated gene transfer. Blood 119, 3038-3041.
4. Flotte T. R., Trapnell B. C., Humphries M., et al. (2011). Phase 2 clinical trial of a recombinant adeno-associated viral vector expressing al-antitrypsin: interim results. Hum. Gene Ther. 22, 1239-1247.
5. Rivera V. M., Ye X., Courage N. L., et al. (1999). Long-term regulated expression of growth hormone in mice after intramuscular gene transfer. Proc. Natl. Acad. Sci. 96, 8657-8662.
6. Lai N. C., Tang T., Gao M. H., et al. (2012). Improved function of the failing rat heart by regulated expression of insulin-like growth factor I via intramuscular gene transfer. Hum. Gene Ther. 23, 255-261
7. Mitrovic V., Seferovic P. M., Simeunovic D., et al. (2006). Haemodynamic and clinical effects of ularitide in decompensated heart failure. Eur. Heart J. 27, 2823-32.
8. Teerlink J. R., Cotter G., Davison B. A. et al. (2013). Serelaxin, recombinant human relaxin-2, for treatment of acute heart failure (RELAX-AHF): a randomised, placebo-controlled trial. Lancet 381, 29-39.
9. Chan W. Y., Frampton C. M., Crozier L G., et al. (2013). Urocortin-2 infusion in acute decompensated heart failure: findings from the UNICORN study (urocortin-2 in the treatment of acute heart failure as an adjunct over conventional therapy). J. Am. Coll. Cardiol. Heart Fail.; 1: 433-441.
10. Davis M. E., Pemberton C. J., Yandle T. G. (2007). Urocortin 2 infusion in human heart failure. Eur. Heart J. 28, 2589-2597.
11. Gheorghiade M., Greene S. J., Ponikowski P., et al. (2013). Haemodynamic effects, safety, and pharmacokinetics of human stresscopin in heart failure with reduced ejection fraction. Eur. J. Heart Fail. 15, 679-689.
12. Wiley K. E., and Davenport A. P. (2004). CRF2 receptors are highly expressed in the human cardiovascular system and their cognate ligands urocortins 2 and 3 are potent vasodilators. Br. J. Pharmacol. 143, 508-514.
13. Davidson S. M., and Yellon D. M. (2009). Urocortin: a protective peptide that targets both the myocardium and vasculature. Pharmacol. Rep. 61, 172-182.
14. Davidson S. M., Rybka A. E., and Townsend P. A. (2009). The powerful cardioprotective effects of urocortin and the corticotropin releasing hormone (CRH) family. Biochem. Pharmacol. 77, 141-150.
15. Bale T. L., Hoshijima M., Gu Y., et al. (2004). The cardiovascular physiologic actions of urocortin II: acute effects in murine heart failure. Proc. Natl. Acad. Sci. USA 101, 3697-3702.
16. Rademaker M. T., Charles C. J., Ellmers L. J., et al. (2011). Prolonged urocortin 2 administration in experimental heart failure: sustained hemodynamic, endocrine, and renal effects. Hypertension 57, 1136-1144.
17. Rivera V. M., Gao G. P., Grant R. L., et al. (2005). Long-term pharmacologically regulated expression of erythropoietin in primates following AAV-mediated gene transfer. Blood 105: 1424-1430.
18. Nathwani A. C., Gray J. T., McIntosh J., et al. (2007). Safe and efficient transduction of the liver after peripheral vein infusion of self-complementary AAV vector results in stable therapeutic expression of human FIX in nonhuman primates. Blood. 109: 1414-1421.
19. Nathwani A. C., Rosales C., McIntosh J., et al. (2011). Long-term safety and efficacy following systemic administration of a self-complementary AAV vector encoding human FIX pseudotyped with serotype 5 and 8 capsid proteins. Mol. Ther. 19, 876-875.
20. Murrey D. A., Naughton B. J., Duncan F. J., et al. (2014). Feasibility and safety of systemic rAAV9-hNAGLU delivery for treating muco-polysaccharidosis IIIB: toxicology, biodistribution, and immunological assessments in primates. Hum. Gene Ther. Clin. Dev. 25, 72-84.
21. Mingozzi F, Meulenberg J J, Hui D J, et al. (2009). AAV-1-mediated gene transfer to skeletal muscle in humans results in dose-dependent activation of capsid-specific T cells. Blood. 114, 2077-2086.
22. Manno C S, Pierce G F, Arruda V R, et al. (2006). Successful transduction of liver in hemophilia by AAV-Factor IX and limitations imposed by the host immune response. Nat. Med. 12. 342-347.
23. Hildinger M., Auricchio A., Gao G., et al. (2001). Hybrid vectors based on adeno-associated virus serotypes 2 and 5 for muscle-directed gene transfer. J. Virol. 75, 6199-6203.
24. De B. P., Heguy A., Hackett N. R., et al. (2006). High levels of persistent expression of al-antitrypsin mediated by the nonhuman primate serotype rh.10 adeno-associated virus despite preexisting immunity to common human adeno-associated viruses. Mol. Ther. 13, 67-76.
25. Fang H., Lai N. C., Gao M. H., et al (2102). Comparison of adeno-associated virus serotypes and delivery methods for cardiac gene transfer. Hum. Gene Ther. Methods 23, 234-241.
26. Boutin S., Monteilhet V., Veron P., et al. (2010). Prevalence of serum IgG and neutralizing factors against adeno-associated virus (AAV) types 1, 2, 5, 6, 8, and 9 in the healthy population: implications for gene therapy using AAV vectors. Hum. Gene Ther. 21, 704-712.
27. Gao M. H., Lai N. C., Miyanohara A., et al. (2013). Intravenous adeno-associated virus serotype 8 encoding urocortin-2 provides sustained augmentation of left ventricular function in mice. Hum. Gene Ther. 24: 777-785.
28. Xiao X., Li J., Samulski R. J. (1998). Production of high-titer recombinant adeno-associated virus vectors in the absence of helper adenovirus. J. Virol. 72, 2224-2232.
29. Gao G., Ou G., Burnham M. S., et al. (2000). Purification of recombinant adeno-associated virus vectors by column chromatography and its performance in vivo. Hum. Gene Ther. 11, 2079-2091.
30. Zolotukhin S., Potter M., Zolotukhin I., et al. (2002). Production and purification of serotype 1, 2, and 5 recombinant adeno-associated viral vectors. Methods 28, 158-167.
31. Bayat H., Swaney J., Ander A., et al. (2002). Progressive heart failure after myocardial infarction in mice. Basic Res. Cardiol. 97, 206-213.
32. Lai N. C., Tang T., Gao M. H., et al. (2008). Activation of cardiac adenylyl cyclase expression increases function of the failing ischemic heart in mice. J. Am. Coll. Cardiol. 51, 1490-1497.
33. Gao M. H., Lai N. C., Roth D. M., et al. (1999). Adenylylcyclase increases responsiveness to catecholamine stimulation in transgenic mice. Circulation 99, 1618-1622.
34. Suarez J, Scott B, Dillmann W H. (2008) Conditional increase in SERCA2a protein is able to reverse contractile dysfunction and abnormal calcium flux in established diabetic cardiomyopathy. Am. J. Physiol. 295: R1439-1445.
35. Gao M. H., Tang T., Guo T. et al. (2008). Adenylyl cyclase type VI increases Akt activity and phospholamban phosphorylation in cardiac myocytes. J. Biol. Chem. 283, 33527-33535.
36. Gao M. H., Tang T., Lai N. C., et al. (2011). Beneficial effects of adenylyl cyclase type 6 (AC6) expression persist using a catalytically inactive AC6 mutant. Mol Pharmacol. 79, 381-388.
37. Bhargava V., Shabetai R., Mathiasen R. A., et al. (1998). Loss of adrenergic control of the force-frequency relation in heart failure secondary to idiopathic or ischemic cardiomyopathy. Am. J. Cardiol. 81, 1130-1137.
38. Hare J. M., Givertz M. M., Creager M. A., et al. (1998). Increased sensitivity to nitric oxide synthase inhibition in patients with heart failure: potentiation of beta-adrenergic inotropic responsiveness. Circulation. 97, 161-166.
39. Swaminathan P. D., Purohit A., Hund T. J., et al. (2012). Calmodulin-dependent protein kinase II: linking heart failure and arrhythmias. Circ Research 110, 1661-1677.
40. Zhang T., Maier L. S., Dalton N. D., et al. (2003). The delta C isoform of CaMKII is activated in cardiac hypertrophy and induces dilated cardiomyopathy and heart failure. Circ. Res. 92, 912-919.
41. He B. J., Joiner M. L., Singh M. V., et al. (2011). Oxidation of CaMKII determines the cardiotoxic effects of aldosterone. Nat. Med. 17, 1610-1618.
42. Sossalla S., Fluschnik N., Schotola H., et al. (2010). Inhibition of elevated Ca2+/calmodulin-dependent protein kinase II improves contractility in human failing myocardium. Circ. Res. 107, 1150-1161.
43. Seguchi O., Seguchi O., Takashima S, et al. (2007). A cardiac myosin light chain kinase regulates sarcomere assembly in the vertebrate heart. J. Clin. Invest. 117: 2812-2824.
44. Chan J. Y., Takeda M., Briggs L. E., et al. (2008). Identification of cardiac-specific myosin light chain kinase. Circ. Res. 102, 571-580.
45. Gu X., Liu X., Xu D., et al. (2010). Cardiac functional improvement in rats with myocardial infarction by up-regulating cardiac myosin light chain kinase with neuregulin. Cardiovasc. Res. 88, 334-343.
46. Ding P., Huang J., Battiprolu P. K., et al. (2010). Cardiac myosin light chain kinase is necessary for myosin regulatory light chain phosphorylation and cardiac performance in vivo. J. Biol. Chem. 285, 40819-40829.
47. Bers D. M. (2002). Cardiac excitation-contraction coupling. Nature; 415: 198-205
48. Wang Z., Zhu T., et al. (2015). Adeno-associated virus serotype 8 efficiently delivers genes to muscle and heart. Nat. Biotechnol. 23, 321-328.

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating, ameliorating or protecting (preventing), slowing the progress of, or reversing: a congestive heart failure (CHF); a type-2 diabetes mellitus (T2DM) and congestive heart failure (CHF); or a diabetes-related cardiac dysfunction in a type 2 diabetic (T2DM), in an individual or a patient,
the method comprising:
(a) (i) providing a urocortin 2 and/or a urocortin 3 polypeptide-encoding nucleic acid or gene operatively linked to a transcriptional regulatory sequence; or an expression vehicle, a vector, a recombinant virus, or equivalent, having contained therein a urocortin 2 and/or a urocortin 3-encoding nucleic acid or gene, or a urocortin 2 and/or a urocortin 3 polypeptide-expressing nucleic acid, transcript or message, and the expression vehicle, vector, recombinant virus, or equivalent can express the urocortin 2 and/or a urocortin 3-encoding nucleic acid, gene, transcript or message in a cell or in vivo; and
(ii) administering or delivering the urocortin 2 and/or a urocortin 3 polypeptide-encoding nucleic acid, gene, transcript or message operatively linked to a transcriptional regulatory sequence, or the expression vehicle, vector, recombinant virus, or equivalent, to the cell, or an individual or a patient in need thereof,
thereby treating, ameliorating or protecting (preventing) the congestive heart failure (CHF); the type-2 diabetes mellitus (T2DM) and congestive heart failure (CHF); or, the diabetes-related cardiac dysfunction in a type 2 diabetic (T2DM), in the individual or patient;
(b) the method of (a), wherein the expression vehicle, vector, recombinant virus, or equivalent is or comprises:
an adeno-associated virus (AAV), a lentiviral vector or an adenovirus vector,
an AAV serotype AAV5, AAV6, AAV8 or AAV9,
a rhesus-derived AAV, or the rhesus-derived AAV AAVrh.10hCLN2,
an AAV capsid mutant or AAV hybrid serotype,
an organ-tropic AAV, optionally, liver-tropic or skeletal muscle-tropic,
wherein optionally the AAV is engineered to increase efficiency in targeting a specific cell type that is non-permissive to a wild type (wt) AAV and/or to improve efficacy in infecting only a cell type of interest,
and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;
(c) the method of (a), wherein the urocortin 2 and/or a urocortin 3-encoding nucleic acid, gene, transcript or message is operatively linked to a regulated or inducible transcriptional regulatory sequence;
(d) the method of (c), wherein the regulated or inducible transcriptional regulatory sequence is a regulated or inducible promoter,
wherein optionally a positive (an activator) and/or a negative (a repressor) modulator of transcription and/or translation is operably linked to the urocortin 2 and/or urocortin 3 polypeptide-encoding nucleic acid, gene, transcript or message;
(e) the method of any of (a) to (d), wherein administering the urocortin 2 and/or urocortin 3 polypeptide-encoding nucleic acid, gene, transcript or message operatively linked to a transcriptional regulatory sequence, or the expression vehicle, vector, recombinant virus, or equivalent, to an individual or a patient in need thereof results in a urocortin 2 and/or urocortin 3 protein being released into the bloodstream or general circulation, or an increased or sustained expression of the urocortin 2 and/or urocortin 3 protein in the cell,
wherein optionally the release or increased or sustained expression of the urocortin 2 and/or urocortin 3 protein is dependent on activation of an inducible promoter, or de-repression of a repressor, operably linked to the urocortin 2 and/or urocortin 3 polypeptide-encoding nucleic acid, gene, transcript or message; or
(f) the method of any of (a) to (e), wherein the disease or condition responsive to an increased urocortin 2 and/or urocortin 3 polypeptide level in vivo is a cardiac contractile dysfunction; a congestive heart failure (CHF); a cardiac fibrosis; a cardiac myocyte disease, dysfunction or apoptosis; a pulmonary hypertension; a heart, skin, liver, lung, muscle, nerve, brain or kidney disease; or, a hemophilia or a Hemophilia B.

2. The method of claim 1, wherein:
(a) the urocortin 2 and/or urocortin 3-encoding nucleic acid or gene operatively linked to the transcriptional regulatory sequence; or the expression vehicle, vector, recombinant virus, or equivalent, is administered or delivered to the individual or a patient in need thereof, by oral, intramuscular (IM) injection, by intravenous (IV) injection, by subcutaneous (SC) or intradermal injection, by intrathecal injection, by intra-arterial (IA) injection, by intracoronary injection, by inhalation, by aerosol, or by a biolistic particle delivery system, or by using a “gene gun”, air pistol or a HELIOS™ gene gun (Bio-Rad Laboratories, Hercules, Calif.); or
(b) the urocortin 2 and/or urocortin 3-encoding nucleic acid or gene operatively linked to the transcriptional regulatory sequence; or the expression vehicle, vector, recombinant virus, or equivalent, is administered or delivered to the individual or a patient in need thereof, by introduction into any tissue or fluid space within the body that is adjacent to or is drained by the bloodstream, such that the encoded protein may be secreted from cells in the tissue and released into the bloodstream.

3. The method of claim 1, wherein:
(a) the individual, patient or subject is administered a stimulus or signal that induces expression of the urocortin 2 and/or a urocortin 3-expressing nucleic acid or gene, or induces or activates a promoter (e.g., operably linked to the urocortin 2 and/or urocortin 3-expressing nucleic acid or gene) that induces expression of the urocortin 2 and/or urocortin 3-expressing nucleic acid or gene;
(b) the individual, patient or subject is administered a stimulus or signal that induces synthesis of an activator of a promoter, optionally a urocortin 2 and/or urocortin 3-expressing nucleic acid or gene-specific promoter (e.g., operably linked to the urocortin 2 and/or urocortin 3-expressing nucleic acid or gene);
(c) the individual, patient or subject is administered a stimulus or signal that induces synthesis of a natural or a synthetic activator of the urocortin 2 and/or urocortin 3-expressing nucleic acid or gene or the urocortin 2 and/or urocortin 3-expressing nucleic acid or gene-specific promoter,
wherein optionally the natural activator is an endogenous transcription factor;
(d) the method of (c), wherein the synthetic activator is a zinc-finger DNA binding protein designed to specifically and selectively turn on an endogenous or exogenous target gene, wherein optionally the endogenous target is a gene urocortin 2 and/or urocortin 3-expressing nucleic acid or gene or an activator of a urocortin 2 and/or urocortin 3-expressing nucleic acid or gene, or an activator of a promoter operatively linked to a urocortin 2 and/or urocortin 3-expressing nucleic acid or gene;
(e) the method of any of (a) to (c), wherein the stimulus or signal comprises a biologic, a light, a chemical or a pharmaceutical stimulus or signal;
(f) the individual, patient or subject is administered a stimulus or signal that stimulates or induces expression of a post-transcriptional activator of a urocortin 2 and/or urocortin 3-expressing nucleic acid or gene, or an activator of a promoter operatively linked to a urocortin 2 and/or urocortin 3-expressing nucleic acid or gene, or
(g) the individual, patient or subject is administered a stimulus or signal that inhibits or induces inhibition of a transcriptional repressor or a post-transcriptional repressor of a urocortin 2 and/or urocortin 3-expressing nucleic acid or gene.

4. The method of claim 5, wherein the chemical or pharmaceutical that induces expression of the urocortin 2 and/or urocortin 3-expressing nucleic acid or gene, or induces expression of the regulated or inducible promoter operatively linked to the urocortin 2 and/or urocortin 3-expressing nucleic acid or gene, is an oral antibiotic, a doxycycline or a rapamycin; or a tet-regulation system using doxycycline is used to induce expression of the urocortin 2 and/or urocortin 3-expressing nucleic acid or gene, or an equivalent thereof.

5. The method of claim 1, wherein the urocortin 2 and/or urocortin 3-expressing nucleic acid or gene or the expression vehicle, vector, recombinant virus, or equivalent, is formulated in a liquid, a gel, a hydrogel, a powder or an aqueous or a saline formulation.

6. The method of claim 1, wherein the urocortin 2 and/or urocortin 3-expressing nucleic acid or gene or the expression vehicle, vector, recombinant virus, or equivalent, is formulated in a vesicle, liposome, nanoparticle or nanolipid particle (NLP).

7. The method of claim 1, wherein the urocortin 2 and/or urocortin 3-expressing nucleic acid or gene or the expression vehicle, vector, recombinant virus, or equivalent, is formulated in an isolated or cultured cell, and optionally the cell is a mammalian cell, a cardiac cell, or a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

8. The method of claim 1, wherein the urocortin 2 and/or urocortin 3-expressing nucleic acid or gene or the expression vehicle, vector, recombinant virus, or equivalent, is formulated as a pharmaceutical or sterile.

9. The method of claim 1, wherein the urocortin 2 and/or urocortin 3-expressing nucleic acid or gene or the expression vehicle, vector, recombinant virus, or equivalent, is formulated or delivered with, on, or in conjunction with a product of manufacture, an artificial organ or an implant.

10. The method of claim 1, wherein the urocortin 2 and/or urocortin 3-expressing nucleic acid or gene or the expression vehicle, vector, recombinant virus, or equivalent expresses a urocortin 2 and/or urocortin 3 polypeptide in vitro or ex vivo.

11. A method for treating, ameliorating or protecting (preventing) an individual or a patient against a urocortin 2 and/or urocortin 3-responsive pathology, disease, illness, or condition, comprising practicing the method of claim 1.

12. A method for treating, ameliorating or protecting (preventing) a diabetes-related cardiac contractile dysfunction; a diabetes-related congestive heart failure (CHF); a diabetes-related cardiac fibrosis; a diabetes-related cardiac myocyte disease, dysfunction or apoptosis; a diabetes-related pulmonary hypertension, comprising practicing the method of claim 1.

13. A method of treating, ameliorating or protecting (preventing) diabetes or pre-diabetes in a patient or an individual comprising:
(a) practicing the method of claim 1; or
(b) administering a urocortin 2 and/or urocortin 3 peptide or polypeptide, or a nucleic acid, gene, message or transcript encoding a urocortin 2 and/or urocortin 3 to an individual or patient in need thereof,
wherein optionally the urocortin 2 and/or urocortin 3 peptide or polypeptide is an isolated, a recombinant, a synthetic and/or a peptidomimetic peptide or polypeptide or variant thereof,
thereby treating, ameliorating or protecting (preventing) the diabetes or pre-diabetes in the patient or individual.

14. A method of treating, ameliorating or protecting (preventing) obesity in a patient or an individual comprising:
(a) practicing the method of claim 1, or
(b) administering a urocortin-2 (UCn-2) peptide or polypeptide, or a nucleic acid, gene, message or transcript encoding a urocortin 2 and/or urocortin 3 to an individual or patient in need thereof,
wherein optionally the urocortin 2 and/or urocortin 3 peptide or polypeptide is an isolated, a recombinant, a synthetic and/or a peptidomimetic peptide or polypeptide or variant thereof,
thereby treating, ameliorating or protecting (preventing) the obesity in the patient or individual.

15. The method of claim 1, wherein the urocortin 2 and/or urocortin 3 urocortin-2 (UCn-2) peptide or polypeptide is formulated in or as a vesicle, liposome, nanoparticle or nanolipid particle (NLP), or is formulated for: oral administration, intramuscular (IM) injection, intravenous (IV) injection, subcutaneous (SC) or intradermal injection, intrathecal injection, intra-arterial (IA) injection, intracoronary injection, inhalation, or administration by aerosol.

16. Use of:
a urocortin 2 and/or a urocortin 3 polypeptide-encoding nucleic acid or gene operatively linked to a transcriptional regulatory sequence;
an expression vehicle, a vector, a recombinant virus, or equivalent, having contained therein a urocortin 2 and/or a urocortin 3-encoding nucleic acid or gene; or
a urocortin 2 and/or a urocortin 3 polypeptide-expressing nucleic acid, transcript or message, and the expression vehicle, vector, recombinant virus, or equivalent that can express the urocortin 2 and/or a urocortin 3-encoding nucleic acid, gene, transcript or message in a cell or in vivo,
in the manufacture of a medicament, or,
said use being, or comprising:
treating, ameliorating or protecting (preventing), slowing the progress of, or reversing, a type-2 diabetes mellitus (T2DM) and congestive heart failure (CHF) in an individual or a patient,
treating, ameliorating or protecting (preventing), slowing the progress of, or reversing, a cardiac contractile dysfunction; a congestive heart failure (CHF); a cardiac fibrosis; a cardiac myocyte disease, dysfunction or apoptosis; a pulmonary hypertension; a heart, skin, liver, lung, muscle, nerve, brain or kidney disease; or, a hemophilia or a Hemophilia B,
treating, ameliorating or protecting or preventing diabetes or pre-diabetes in a patient or an individual, or
treating, ameliorating or protecting or preventing obesity in a patient or an individual,
wherein optionally the expression vehicle, vector, recombinant virus, or equivalent is or comprises:
an adeno-associated virus (AAV), a lentiviral vector or an adenovirus vector,
an AAV serotype AAV5, AAV6, AAV8 or AAV9,
a rhesus-derived AAV, or the rhesus-derived AAV AAVrh.10hCLN2,
an AAV capsid mutant or AAV hybrid serotype,
an organ-tropic AAV, optionally, liver-tropic or skeletal muscle-tropic,
wherein optionally the AAV is engineered to increase efficiency in targeting a specific cell type that is non-permissive to a wild type (wt) AAV and/or to improve efficacy in infecting only a cell type of interest,
and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;
wherein optionally the urocortin 2 and/or a urocortin 3-encoding nucleic acid, gene, transcript or message is operatively linked to a regulated or inducible transcriptional regulatory sequence;
wherein optionally the regulated or inducible transcriptional regulatory sequence is a regulated or inducible promoter,
wherein optionally a positive (an activator) and/or a negative (a repressor) modulator of transcription and/or translation is operably linked to the urocortin 2 and/or urocortin 3 polypeptide-encoding nucleic acid, gene, transcript or message.

17. A urocortin 2 and/or a urocortin 3 polypeptide-encoding nucleic acid or gene operatively linked to a transcriptional regulatory sequence; or,
an expression vehicle, a vector, a recombinant virus, or equivalent, having contained therein a urocortin 2 and/or a urocortin 3-encoding nucleic acid or gene; or,
a urocortin 2 and/or a urocortin 3 polypeptide-expressing nucleic acid, transcript or message, and the expression vehicle, vector, recombinant virus, or equivalent that can express the urocortin 2 and/or a urocortin 3-encoding nucleic acid, gene, transcript or message in a cell or in vivo,
for use in the manufacture of a medicament, or,
for use in:
treating, ameliorating or protecting (preventing), slowing the progress of, or reversing, a type-2 diabetes mellitus (T2DM) and congestive heart failure (CHF) in an individual or a patient,
treating, ameliorating or protecting (preventing), slowing the progress of, or reversing, a cardiac contractile dysfunction; a congestive heart failure (CHF); a cardiac fibrosis; a cardiac myocyte disease, dysfunction or apoptosis; a pulmonary hypertension; a heart, skin, liver, lung, muscle, nerve, brain or kidney disease; or, a hemophilia or a Hemophilia B,
treating, ameliorating or protecting or preventing diabetes or pre-diabetes in a patient or an individual, or
treating, ameliorating or protecting or preventing obesity in a patient or an individual,
comprising providing and administering or delivering the:
urocortin 2 and/or a urocortin 3 polypeptide-encoding nucleic acid or gene operatively linked to a transcriptional regulatory sequence;
expression vehicle, a vector, a recombinant virus, or equivalent, having contained therein a urocortin 2 and/or a urocortin 3-encoding nucleic acid or gene; or
urocortin 2 and/or a urocortin 3 polypeptide-expressing nucleic acid, transcript or message, and the expression vehicle, vector, recombinant virus, or equivalent that can express the urocortin 2 and/or a urocortin 3-encoding nucleic acid, gene, transcript or message in a cell or in vivo,
to a cell of the subject, or to a subject in need thereof;
wherein optionally the expression vehicle, vector, recombinant virus, or equivalent is or comprises:
an adeno-associated virus (AAV), a lentiviral vector or an adenovirus vector,
an AAV serotype AAV5, AAV6, AAV8 or AAV9,
a rhesus-derived AAV, or the rhesus-derived AAV AAVrh.10hCLN2,
an AAV capsid mutant or AAV hybrid serotype,
an organ-tropic AAV, optionally, liver-tropic or skeletal muscle-tropic,
wherein optionally the AAV is engineered to increase efficiency in targeting a specific cell type that is non-permissive to a wild type (wt) AAV and/or to improve efficacy in infecting only a cell type of interest,
and optionally the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid;
wherein optionally the urocortin 2 and/or a urocortin 3-encoding nucleic acid, gene, transcript or message is operatively linked to a regulated or inducible transcriptional regulatory sequence;
wherein optionally the regulated or inducible transcriptional regulatory sequence is a regulated or inducible promoter,
wherein optionally a positive (an activator) and/or a negative (a repressor) modulator of transcription and/or translation is operably linked to the urocortin 2 and/or urocortin 3 polypeptide-encoding nucleic acid, gene, transcript or message.

18. A method for treating, ameliorating or protecting (preventing) a congestive heart failure (CHF), or the symptoms of congestive heart failure (CHF), in a subject or individual in need thereof, comprising:
(a) delivering to a subject or individual in need thereof a nucleic acid sequence encoding a urocortin 2 polypeptide,
thereby treating or ameliorating congestive heart failure (CHF) in the subject subject or individual in need thereof;
(b) the method of (a), wherein the nucleic acid sequence is in (e.g., contained within) a vector;
(c) the method of (b), wherein the vector is a viral vector;
(d) the method of (c), wherein the vector is an adeno-associated virus (AAV);
(e) the method of (d), wherein the AAV is a serotype AAV8;
(f) the method of any of (a) to (e), wherein the subject or individual in need thereof has a type 2 diabetes (T2DM);
(g) the method of any of (a) to (f), wherein the nucleic acid sequence is administered by intravenous injection (IV) or intramuscularly.
